(19) United States
(12) Patent Application Publication
(10) Pub. No.: US 2024/0069023 A1
(43) Pub. Date: Feb. 29, 2024

(54) DETECTION OF ACE2 IgM AUTOANTIBODIES AS MARKERS OF SEVERITY AND MECHANISM IN COVID19 PATIENTS

(71) Applicant: The Johns Hopkins University, Baltimore, MD (US)

(72) Inventors: Antony Rosen, Pikesville, MD (US); Livia A. Casciola-Rosen, Pikesville, MD (US)

(21) Appl. No.: 18/030,465

(22) PCT Filed: Oct. 12, 2021
(86) PCT No.: PCT/US2021/054571

§ 371 Date: Apr. 5, 2023

Related U.S. Application Data
(60) Provisional application No. 63/090,392, filed on Oct. 12, 2020.

(51) Int. Cl. G01N 33/569; G01N 33/564; G01N 33/543

(52) U.S. Cl. CPC G01N 33/56983; G01N 33/564; G01N 33/54388; G01N 2333/165; G01N 2333/948; G01N 2469/10

(57) ABSTRACT

Provided herein are methods for diagnosing the probability of extended duration of severe COVID19 symptoms in a subject infected with a SARS-CoV2 virus or suspected of being infected with the SARS-CoV2 virus, the method including (a) providing a biological sample from the subject; and (b) detecting the presence of an anti-ACE2 IgM antibody in the biological sample, thereby diagnosing the probability of extended duration of severe COVID19 symptoms in the subject.

### CROSS-REFERENCE TO RELATED APPLICATION

This applicant claims priority to U.S. Provisional Application No. 63/090,392, filed on Oct. 12, 2020, which is incorporated by reference herein in its entirety.

### BACKGROUND OF THE INVENTION

COVID19 is a global pandemic caused by the novel coronavirus SARS-Cov2¹. It is a highly infectious pathogen, and continues to have a massive global impact since its recognition in Wuhan in late 2019, with at least 235 million infections, >4 million deaths and massive economic disruption across the world. While most infections appear to be self-limited, 15-20% of symptomatic individuals become hospitalized, and 5-10% require admission to ICUs^{2,3}. Mortality rates of hospitalized patients in the U.S. range between 13 and 28%. Growing evidence suggests that some of the severe COVID clinical features represent damage induced by activation of the immune and inflammatory responses initiated by the virus^{4,5,6}. In addition to frequent acute respiratory distress syndrome (ARDS), there is also evidence of vasculopathy^{7,8}, clotting^{9,10}, and cardiovascular complications¹¹ whose mechanism is presently unclear, but in which complement activation has been implicated^{12,9}.

The recent finding that low-dose dexamethasone has a beneficial effect on mortality in a subgroup of patients with severe COVID requiring ventilation has suggested that uncontrolled inflammatory mechanisms might play an apical role in mediating disease severity in some patients with this disease¹³. Understanding these mechanisms is therefore a high priority, particularly if they might be rapidly addressed therapeutically with additional off-the-shelf approaches.

Angiotensin converting enzyme 2 (ACE2) has been known as a potential autoantigen in COVID. Its expression is enhanced in lung (epithelial and endothelial cells) and heart (endothelial cells)¹⁵, and hypomorphic ACE2 function has been implicated in adverse outcomes in models of ARDS¹⁴. Additionally, SARS-CoV2 spike protein binds with higher affinity (5-20 fold higher) to ACE2 than the other coronaviruses which also bind to this host receptor¹⁵.

As such, there is an unmet need for quick diagnosis and detection of markers of severe COVID-19 infection in patients.

### SUMMARY OF THE INVENTION

Provided herein are methods for diagnosing the probability of extended duration of severe COVID19 symptoms in a subject infected with a SARS-CoV2 virus or suspected of being infected with the SARS-CoV2 virus, the method comprising: (a) providing a biological sample from the subject; and (b) detecting the presence of an anti-ACE2 IgM antibody in the biological sample, thereby diagnosing the probability of extended duration of severe COVID19 symptoms in the subject.

In some embodiments, the extended duration of severe COVID19 symptoms is about 44 days.

Also provided herein are methods for diagnosing the probability of severe COVID19 symptoms in a subject infected with a SARS-CoV2 virus or suspected of being infected with the SARS-CoV2 virus, the method comprising: (a) contacting a biological sample from the subject with an antibody that binds to an anti-ACE2 IgM antibody in the biological sample, producing an antibody-anti-ACE2 IgM antibody complex; and (b) detecting the presence of the antibody-anti-ACE2 IgM antibody complex, thereby diagnosing the probability of severe COVID19 symptoms in the subject.

In some embodiments, the method comprises an immunoassay to detect the presence of the antibody-anti-ACE2 IgM antibody complex. In some embodiments, the immunoassay is an ELISA assay.

Also provided herein are methods for treating a subject infected with a SARS-CoV2 virus or suspected of being infected with the SARS-CoV2 virus, the method comprising: (a) providing a biological sample from the subject; (b) detecting the presence of an anti-ACE2 IgM antibody in the biological sample, wherein the presence of the anti-ACE2 IgM antibody subject indicates the probability of severe COVID19 symptoms; and (c) administering to the subject a COVID19 treatment, thereby treating the subject infected with the SARS-CoV2 virus.

In some embodiments, a severe COVID19 symptom comprises one or more of: high fever, severe cough, shortness of breath, difficulty breathing, fatigue, muscle aches, headache, loss of taste or smell, congestion, nausea, persistent pain or pressure in the chest, confusion, inability to wake or stay awake, delirium, seizures, or stroke.

Also provided herein are methods of screening for a clinically effective therapeutic agent to treat a subject infected with a SARS-CoV2 virus, the method comprising: (a) providing a biological sample from the subject; (b) detecting the presence of an anti-ACE2 IgM antibody in the biological sample; and (c) screening for a clinically effective therapeutic agent to administer to the subject as a COVID19 treatment.

In some embodiments, the COVID19 treatment comprises an immunosuppressing agent, an anti-SARS-CoV2 monoclonal antibody, an antiviral agent, supplemental oxygen, or mechanical ventilatory support. In some embodiments, the biological sample is a blood sample. In some embodiments, the presence of the anti-ACE2 IgM antibody is detected at the onset of severe disease in the subject.

Also provided herein are kits comprising: (a) a lateral flow device comprising a first pad, a second pad, and a membrane, wherein the first pad receives a biological sample from a subject having a SARS-CoV2 infection, the second pad comprises an antibody that binds to an anti-ACE2 IgM antibody, and the membrane that reacts to the antibody bound to the anti-ACE2 IgM antibody; and (b) instructions to use the kit to detect the anti-ACE2 IgM antibody in the biological sample.

Also provided herein are lateral flow devices adapted to perform the method of claim 3 for detecting an anti-ACE2-IgM antibody in a biological sample of a subject infected with a SARS-CoV2 virus,

In some embodiments, the biological sample is a blood sample.

Also provided herein are dipstick assays to perform the method of claim 3 for detecting an anti-ACE2-IgM antibody in a biological sample of a subject infected with a SARS-CoV2 virus.

In some embodiments, the biological sample is a blood sample.

### BRIEF DESCRIPTION OF THE DRAWINGS

FIGS. 1A-1B Anti-ACE2 IgM antibodies in COVID-19 patients. FIG. 1A. Anti-ACE2 IgM ELISAs were performed as described in the Methods section. In the Discovery cohort (left panel), 8/66 patients with COVID19 were positive for anti-ACE2 IgM antibodies. Of these, significantly more were in the WHO 6-8 group compared to the WHO 3-5 group (p=0.0084, Fisher's exact test). To expand the discovery findings, an additional 52 COVID-19 patients were assayed (“Expanded discovery”, right panel); the frequency of anti-ACE2 IgM in these patients was similar to the initial group (12/52). Data from the combined cohorts (N=118) is shown in FIG. 1A. FIG. 1B. Anti-ACE2 IgM ELISAs were performed using serial serum dilutions (1:100 to 1:3,200 range). Data obtained from four different patients is shown in the left panel, each assayed using serum from a single bleed. Data from a fifth patient is shown in the right panel, using serum made from blood draws on 4 different days. Area under the curve (“AUC”) plots are shown in both panels.

FIGS. 2A-2B Anti-ACE2 IgM antibodies are found in patients with COVID-19. FIG. 2A: Antibodies were assayed by ELISA in the combined COVID cohort (N=118 patients). Left panel: the number of patients with and without anti-ACE2 IgM antibodies is shown grouped by disease severity. 27.2% of severe patients were anti-ACE2 positive compared to 3.8% with moderate COVID (p=0.0009; Fisher's exact test). In the center and right panels, data from anti-ACE2 IgM and IgG ELISA assays, respectively, is presented as corrected OD 450 absorbance units. This data was obtained on all the COVID patients presented in the left panel, as well as from 30 healthy controls. Red dots in the IgG panel denote samples that also have anti-ACE2 IgM antibodies. The horizontal line on each plot represents the cutoff for assigning a positive antibody status. FIG. 2B: Longitudinal analysis of anti-ACE2 IgM antibodies. For all those anti-ACE2 IgM-positive patients with multiple banked sera available, anti-ACE2 IgM and IgG antibodies were quantitated over time. Red and blue lines on each plot denote anti-ACE2 IgM and IgG antibodies, respectively. Solid black bars represent steroid treatment periods. Additional examples are shown in FIG. 5.

FIGS. 3A-3F Clinical features of anti-ACE2 IgM-positive COVID-19 patients (N=20) compared to those that do not have these antibodies (N=98). FIGS. 3A-3E: Age, BMI, sex, temperature and C reactive protein (CRP) levels were compared between the anti-ACE2 IgM-positive and negative COVID patient groups. Red and blue colors denote anti-ACE2 IgM-antibody positive and negative status, respectively. FIG. 3F: Anti-ACE2 IgM antibodies are detected in COVID-19 patients but not in other infectious and autoimmune disease controls.

FIG. 4: MC on stained paraffin sections from autopsy of COVID patient's lungs. (left panel) A section of the left upper lobe of the lung shows a widened interstitium with capillaries showing reactive endothelium. There are hyaline membranes lining alveolar spaces, consistent with the exudative phase of diffuse alveolar damage (acute lung injury). (Hematoxylin & eosin staining, 100×). (right panel) Anti-IgM immunohistochemical staining highlights capillary endothelium in the same area.

FIG. 5 Longitudinal analysis of anti-ACE2 IgM antibodies in patients hospitalized with severe COVID-19. For all those anti-ACE2 IgM-positive patients with multiple banked sera, anti-ACE2 IgM and IgG antibodies were quantitated over time. Red and blue lines on each plot denote anti-ACE2 IgM and IgG antibodies, respectively. Additional examples are shown in FIG. 2B.

FIGS. 6A-6D Properties of anti-ACE2 IgM antibodies. (FIGS. 6A & 6B): IgM isolated from SARS-Cov-2-infected patients, but not healthy donors, binds to ACE2. FIG. 6A: Kinetic traces of the binding interactions between immobilized human ACE2 and purified IgM, as determined by biolayer interferometry. Percentages represent twofold dilutions of IgM from patient CV-1 and Control B. FIG. 6B: Equilibrium binding titrations. Normalized responses at the indicated concentrations of purified IgM from the donors shown in (6A) are plotted (see FIG. 5A-C for data obtained from another patient and control). Equilibrium dissociation constants (KD) are provided. FIG. 6C: Anti-ACE2 IgM antibodies do not inhibit ACE2 activity. ACE2 activity, in the presence or absence of IgM from patient CV-1 or Control B, was measured using a fluorescent substrate in a time course assay. The positive control was ACE2 alone, and the negative control was ACE2 plus ACE2 inhibitor (see FIG. 8D for data obtained from another patient and control). FIG. 6D: Complement activation by IgM antibodies to ACE2. Dynabeads containing immune complexes of ACE2 and purified IgM from healthy control (HC) or anti-ACE2 IgM from patients with COVID-19 (CV) were incubated with human complement. Deposition of Clq and C3 was visualized by immunoblotting. Detection of ACE2 is shown as loading control.

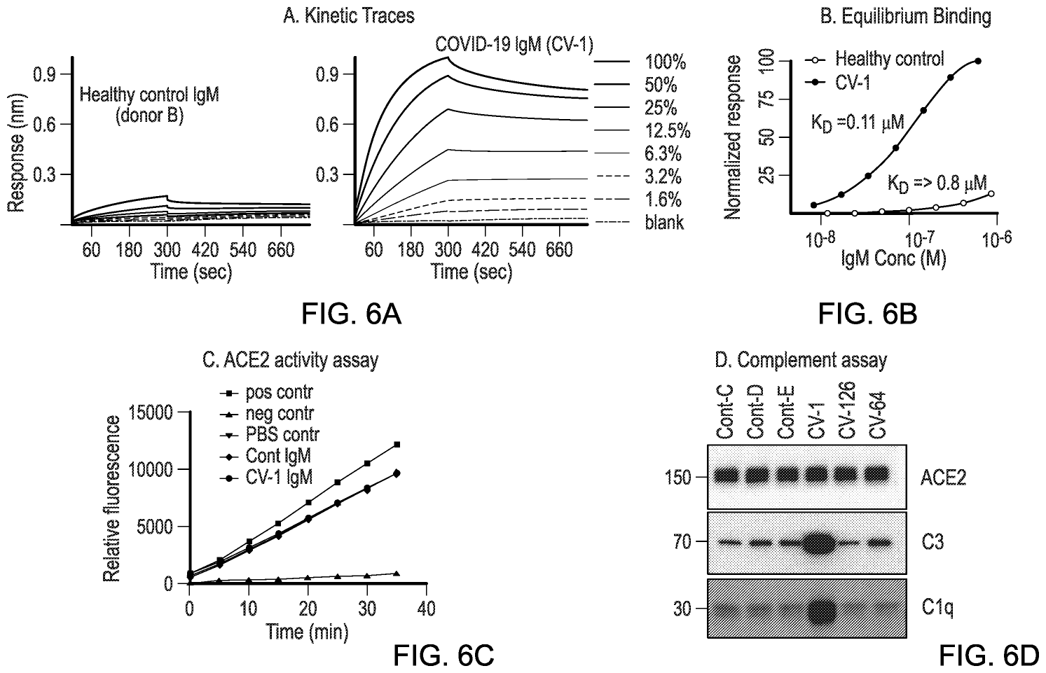

FIGS. 7A-7B Antibodies against SARS-CoV2 S-protein in anti-ACE2-positive COVID-19 patients. FIG. 7A (left and right panels): Anti-SARS-CoV2 S-protein IgG antibodies were assayed by ELISA. Patients are shown grouped by disease severity in (left panel), and by anti-ACE2 IgM antibody status in (right panel). The mean ODs of anti-S antibodies were significantly higher in patients with severe compared to mild COVID (P<0.0001, Chi-squared). The median anti-S-antibody level was significantly higher in anti-ACE2 IgM-positive patients compared to anti-ACE2 IgM-negatives (P=0.028, Mann-Whitney test). FIG. 7B: Anti-S and -RBP antibodies assayed by the CoronaChek point of care assay. 8/8 (100%) of anti-ACE2 IgM-positive patients had a positive IgG result, compared to only 31/58 (53.4%) of anti-ACE2 IgM-negative patients (p=0.017, Fisher's exact test). Red and blue denote anti-ACE2 IgM antibody-positive and -negative patients, respectively.

FIGS. 8A-8E Properties of anti-ACE2 IgM antibodies. (FIGS. 8A-8C): IgM isolated from SARS-Cov-2-infected patients, but not healthy donors, binds to ACE2. FIG. 8A: Kinetic traces of the binding interactions between immobilized human ACE2 and purified IgM, as determined by biolayer interferometry. Percentages represent twofold dilutions of IgM from patient CV-64 and Control A. FIG. 8B: Equilibrium binding titrations. Normalized responses at the indicated concentrations of purified IgM from the donors shown in (FIG. 8A) are plotted (see FIGS. 6A-6B for similar data obtained from COVID patient CV-1 and control B). Equilibrium dissociation constants (KD) are provided. FIG. 8C: Quantitation of the data obtained in FIG. 8A-8B, and FIG. 6A-6B. FIG. 8D: Anti-ACE2 IgM antibodies do not inhibit ACE2 activity. ACE2 activity, in the presence or absence of IgM from patient CV-64 or Control A, was measured using a fluorescent substrate in a time course assay. The positive control was ACE2 alone, and the negative control was ACE2 plus ACE2 inhibitor (see FIG. 6C for similar data obtained from patient CV-1 and control B). FIG. 8E. Complement activation by IgM antibodies to ACE2. Dynabeads containing immune complexes of ACE2 and purified IgM from healthy control (HC) or anti-ACE2 IgM from patients with COVID-19 (CV) were incubated with human complement. Deposition of Clq and C3 was visualized by immunoblotting. Detection of ACE2 is shown as loading control.

FIG. 9 Graph showing the temperatures of patients when we restrict the analysis to the severe IgM-positive patients above and compare them to all severe COVID-19 patients from the CROWN Registry for whom IgM status were unknown (IgM-positive: mean=37.53, S³=0.64, N=721 on M=18 unique patients, IgM-unknown: mean=37.11, S²=0.59, N=14827 on M=473 unique patients; chisq=19.98, p=0.0005 from linear mixed-effects model Wald test with 4 degrees of freedom.

### DETAILED DESCRIPTION OF THE INVENTION

COVID-19 is a global pandemic caused by the novel coronavirus SARS-CoV-2¹. While most infections appear to be self-limited, 15-20% of symptomatic individuals become hospitalized, and 5-10% require admission to ICUs^{2,3}. Growing evidence suggests that some of the severe clinical features of COVID-19 represent damage of blood vessels induced by activation of the host immune and inflammatory response⁴⁻⁴, although the specific vascular targets of this response remain unknown. It has been shown that IgM autoantibodies that recognize angiotensin converting enzyme-2 (ACE2), the receptor for the SARS-CoV-2 spike protein known to be expressed on blood vessel endothelium, are associated with severe COVID-19. Unexpectedly, these ACE2-reactive IgM do not undergo class-switching to IgG and are unmutated, indicating that this anti-ACE2 response is T-independent. Anti-ACE2 IgM activate complement and initiate changes in endothelial cells in microvessels, suggesting that they contribute to the striking angiocentric pathology and dysfunction of COVID-19. The methods described herein identify a therapeutically tractable, mechanism-based biomarker that is strongly associated with severe clinical outcomes in SARS-CoV-2 infection.

Described herein are method for diagnosing the probability of severe COVID19 symptoms in a subject infected with a SARS-CoV2 virus, the method including (a) providing a biological sample from the subject; and (b) detecting the presence of an anti-ACE2 IgM antibody in the biological sample, thereby diagnosing the probability of severe COVID19 symptoms in the subject.

Various non-limiting aspects of these methods are described herein, and can be used in any combination without limitation. Additional aspects of various components of methods for identifying the presence or absence of a mutation and methylation are known in the art.

The use of the terms “a” and “an” and “the” and similar referents in the context of describing the invention are to be construed to cover both the singular and the plural, unless otherwise indicated herein or clearly contradicted by context.

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Method for Diagnosing the Probability of Severe COVID19 Symptoms or Extended Duration of Severe COVID19 Symptoms

Described herein are methods for diagnosing the probability of extended duration of severe COVID19 symptoms in a subject infected with a SARS-CoV2 virus or suspected of being infected with the SARS-CoV2 virus, the method including (a) providing a biological sample from the subject; and (b) detecting the presence of an anti-ACE2 IgM antibody in the biological sample, thereby diagnosing the probability of extended duration of severe COVID19 symptoms in the subject. Also described herein are methods for diagnosing the probability of severe COVID19 symptoms in a subject infected with a SARS-CoV2 virus or suspected of being infected with the SARS-CoV2 virus, the method including (a) contacting a biological sample from the subject with an antibody that binds to an anti-ACE2 IgM antibody in the biological sample, producing an antibody-anti-ACE2 IgM antibody complex; and (b) detecting the presence of the antibody-anti-ACE2 IgM antibody complex, thereby diagnosing the probability of severe COVID19 symptoms in the subject.

It will be understood by those of ordinary skill in the art that the detection of IgM antibodies in the serum of a COVID19 patient is correlative with those subject high in the World Health Organization (WHO) ordinal categories of 1 to 8, e.g. above level 5.

As used herein, the WHO scale is an 8-point ordinal scale ranging from ambulatory (1=asymptomatic, 2=mild limitation in activity), to hospitalized with mild-moderate disease (3=room air, 4=nasal cannula or facemask oxygen), hospitalized with severe disease (5=high flow nasal canula (HFNC) or non-invasive positive pressure ventilation (NIPPY), 6=intubation and mechanical ventilation, 7=intubation and mechanical ventilation and other signs of organ failure (hemodialysis, vasopressors, extracorporeal membrane oxygenation (ECMO)), and 8=death. For this study adjacent WHO classes were combined, and the inpatient population was divided into two groups according to maximum WHO severity: patients who did not require mechanical ventilation (WHO class 3-5); those who required mechanical ventilation with our without additional support, such as intravenous pressors, continuous renal replacement therapy (CRRT) and/or extracorporeal membrane oxygenation (ECMO) who survived (WHO classes 6-7) or died (WHO class 8). Identification of subjects having these antibodies will allow clinicians to properly triage treatments which may lower the immune response in these subjects and prevent organ damage related to inflammation.

As used herein, the term “diagnosing” or “diagnosis” refers to a predictive process in which the presence, absence, severity or course of treatment of a disease, disorder or other medical condition is assessed. In some embodiments, diagnosis also includes predictive processes for determining the outcome resulting from a treatment. Likewise, the term “diagnosing,” refers to the determination of whether a sample specimen exhibits one or more characteristics of a condition or disease. The term “diagnosing” includes establishing the presence or absence of, for example, a target antigen or reagent bound targets, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease.

As used herein, the term “SARS coronavirus infection” or “infected with a SARS-CoV2 virus” refers to a subject who is infected with one or more coronaviruses (CoVs). CoVs are enveloped positive-sense RNA viruses, are characterized by club-like spikes that project from their surface, an unusually large RNA genome, and a unique replication strategy. Coronaviruses cause a variety of diseases in mammals and birds ranging from enteritis in cows and pigs and upper respiratory disease chickens to potentially lethal human respiratory infections.

The initial attachment of the CoV virion to the host cell is initiated by interactions between the spike (S) protein and its receptor. The sites of receptor binding domains (RBD) within the S1 region of a coronavirus S protein vary depending on the virus, with some having the RBD at the N-terminus of S1 (MHV) while others (SARS-CoV) have the RBD at the C-terminus of S1. The S-protein/receptor interaction is the primary determinant for a coronavirus to infect a host species and also governs the tissue tropism of the virus. In some embodiments, coronaviruses utilize peptidases as their cellular receptor. It is unclear why peptidases are used, as entry occurs even in the absence of the enzymatic domain of these proteins. In some embodiments, coronaviruses utilize aminopeptidase N (APN) as their receptor, SARS-CoV, SARS-CoV-2, and HCoV-NL63 use angiotensin-converting enzyme 2 (ACE2) as their receptor, wherein MHV enters through CEACAM1, and the recently identified MERS-CoV binds to dipeptidyl-peptidase 4 (DPP4) to gain entry into human cells. In some embodiments, the subject is infected with SARS-CoV-2, which causes the COVID-19 disease.

As used herein, the term “COVID19 symptom” can refer to physical or medical problems that a subject experiences that may indicate the COVID19 disease or condition. In some embodiments, a COVID19 symptom can include fever, chills, shortness of breath, fatigue, muscle aches, loss of taste or smell, headache, sore throat, congestion, runny nose, nausea, vomiting, or diarrhea. In some embodiments, a COVID19 symptom can be a mild symptom. In some embodiments, a COVID19 symptom can be a severe symptom. In some embodiments, a severe COVID19 symptom comprises one of more of: high fever, severe cough, shortness of breath, difficulty breathing, fatigue, muscle aches, headache, loss of taste or smell, congestion, nausea, persistent pain or pressure in the chest, confusion, inability to wake or stay awake, delirium, seizures, or stroke.

As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “an anti-ACE2 IgM antibody” or “IgM antibodies directed against ACE2” refers to IgM antibodies generated by the immune system of the subject infected with SARS-CoV2 virus which bind with specificity to the ACE2 receptor protein in a sample from the subject.

As used herein, the terms “sample,” “patient sample,” “biological sample,” and the like, encompass a variety of sample types obtained from a patient, individual, or subject and can be used in a diagnostic, prognostic or monitoring assay. In some embodiments, a biological sample may be obtained from a healthy subject, or a diseased patient including, for example, a patient having associated symptoms of COVID19 disease. Moreover, a sample obtained from a patient can be divided and only a portion may be used for diagnosis, prognosis or monitoring. Further, the sample, or a portion thereof, can be stored under conditions to maintain sample for later analysis. The definition specifically encompasses blood and other liquid samples of biological origin (including, but not limited to, peripheral blood, serum, plasma, urine, saliva, amniotic fluid, stool and synovial fluid), solid tissue samples such as a biopsy specimen or tissue cultures or cells derived therefrom and the progeny thereof. In a specific embodiment, a sample comprises a salivary gland sample. In another embodiment, a sample of muscle tissue is used. In other embodiments, a sample comprises a blood or serum sample. The definition also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry. In some embodiments, the biological sample can be a blood sample.

As used herein, the terms “providing a sample” and “providing a biological sample” are used interchangeably and mean to provide or obtain a biological sample for use in methods described herein. In some embodiments, this will be done by removing a sample of cells from a subject, but can also be accomplished by using previously isolated cells (e.g., isolated by another person, at another time, and/or for another purpose), or by performing the methods described herein in vivo. In some embodiments, archival tissues, having treatment or outcome history, can also be used.

As used herein, the term “antibody” or “antibody agent” can refer to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies, polyclonal antibodies, and fragments thereof. In some embodiments, an antibody agent may include one or more sequence elements are humanized, primatized, chimeric, etc., as is known in the art. In many embodiments, the term “antibody” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE, or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody is or comprises a polypeptide whose amino acid sequence includes structural elements recognized by those skilled in the art as an immunoglobulin variable domain. In some embodiments, an antibody is a polypeptide protein having a binding domain which is homologous or largely homologous to an immunoglobulin-binding domain.

Typically, an immunoglobulin has a heavy and light chain. Each heavy and light chain contains a constant region and a variable region, (the regions are also known as “domains”). Light and heavy chain variable regions contain four framework” regions interrupted by three hypervariable regions, also called complementarity-determining regions (CDRs). References to “VH” or a “VH” refer to the variable region of an immunoglobulin heavy chain of an antibody, including the heavy chain of an Fv, scFv, or Fab. References to “VL” or a “VL” refer to the variable region of an immunoglobulin light chain, including the light chain of an Fv, scFv, dsFv or Fab.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The term “fully human antibody” refers to an immunoglobulin comprising human hypervariable regions in addition to human framework and constant regions. Such antibodies can be produced using various techniques known in the art. For example in vitro methods involve use of recombinant libraries of human antibody fragments displayed on bacteriophage (e.g., McCafferty et al., 1990, Nature 348:552-554; Hoogenboom & Winter, J. Mol. Biol. 227:381 (1991); and Marks et al., J. Mol. Biol. 222:581 (1991)), yeast cells (Boder and Wittrup, 1997, Nat Biotechnol 15:553-557), or ribosomes (Hanes and Pluckthun, 1997, Proc Natl Acad Sci USA 94:4937-4942). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 6,150,584, 5,545,807; 5,545,806; 5,625,126; 5,633,425; 5,661,016, and in the following scientific publications: (e.g., Jakobavits, Adv Drug Dehv Rev. 31:33-42 (1998), Marks et al., Bio/Technology (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance See, e.g., Epitope Mapping Protocols in Methods in Molecular Biology, Vol. 66, Glenn E. Morris, Ed (1996).

In some embodiments, a subject exhibiting anti-ACE2 IgM antibodies will suffer or is predicted to suffer from an extended duration of severe COVID19 symptoms. In some embodiments, the extended duration of severe COVID19 symptoms can be more than two weeks (e.g., three weeks, four weeks, five weeks, six weeks, seven weeks, or eight weeks). In some embodiments, the extended duration of severe COVID19 symptoms can be more than one month (e.g., two months, three months, four months, five months, or six months). In some embodiments, the extended duration of severe COVID19 symptoms can be about 44 days (e.g., about 15 days, about 20 days, about 25 days, about 30 days, about 35 days, about 40 days, about 45 days, about 50 days, about 55 days, or about 60 days).

Also disclosed herein are methods for diagnosing the probability of severe COVID19 symptoms in a subject infected with a SARS-CoV2 virus or suspected of being infected with the SARS-CoV2 virus, the method including (a) contacting a biological sample from the subject with an antibody that binds to an anti-ACE2 IgM antibody in the biological sample, producing an antibody-anti-ACE2 IgM antibody complex; and (b) detecting the presence of the antibody-anti-ACE2 IgM antibody complex, thereby diagnosing the probability of severe COVID19 symptoms in the subject.

In some embodiments, antibodies can be used to detect anti-ACE2-IgM antibodies. The detection and/or quantification of anti-ACE2-IgM antibodies can be accomplished using any of a number of well recognized immunological binding assays. A general overview of the applicable technology can be found in Harlow & Lane, Antibodies: A Laboratory Manual (1988) and Harlow & Lane, Using Antibodies (1999). Other resources include see also Methods in Cell Biology: Antibodies in Cell Biology, volume 37 (Asai, ed. 1993); Basic and Clinical Immunology (Stites & Ten, eds., 7th ed. 1991, and Current Protocols in Immunology (Coligan, et al. Eds, John C. Wiley, 1999-present). In some embodiments, immunological binding assays can use either polyclonal or monoclonal antibodies. It will be understood that these antibodies can be detected using a variety of assays for detection known in the art.

In some embodiments, assays for detecting anti-ACE2-IgM antibodies can include noncompetitive assays (e.g., sandwich assays). In some embodiments, formats for detecting anti-ACE2-IgM antibodies can include immunoblots, which are used to detect and quantify the presence of anti-ACE2-IgM antibodies in a sample. In some embodiments, other assay formats for detecting anti-ACE2-IgM antibodies can include liposome immunoassays (LIA), which use liposomes designed to bind specific molecules (e.g., antibodies) and release encapsulated reagents or markers, which are then detected according to standard techniques (see Monroe et al., Amer. Clin. Prod. Rev. 5:34-41 (1986)).

In some embodiments, immunoassays for detecting anti-ACE2-IgM antibodies use a labeling agent to specifically bind to and label the complex formed by the antibody and antigen. The labeling agent may itself be one of the moieties comprising the antibody/antigen complex. In some embodiments, the labeling agent may be labeled for anti-ACE2-IgM antibodies. Alternatively, the labeling agent may be a third moiety, such as a secondary antibody, that specifically binds to the antibody/antigen complex (a secondary antibody is typically specific to antibodies of the species from which the first antibody is derived). In some embodiments, other proteins capable of specifically binding immunoglobulin constant regions, such as protein A or protein G may also be used as the labeling agent. In some embodiments, the labeling agent can be modified with a detectable moiety, such as biotin, to which another molecule can specifically bind, such as streptavidin. A variety of detectable moieties are well known to those skilled in the art.

The particular label or detectable group used in the assay is not a critical aspect of the disclosure, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent compounds (e.g., fluorescein isothiocyanate, Texas red, rhodamine, fluorescein, and the like), radiolabels, enzymes (e.g., horse radish peroxidase, alkaline phosphatase and others commonly used in an ELISA), streptavidin/biotin, and colorimetric labels such as colloidal gold or colored glass or plastic beads (e.g., polystyrene, polypropylene, latex, etc.). Chemiluminescent compounds may also be used. For a review of various labeling or signal producing systems that may be used, see U.S. Pat. No. 4,391,904.

In some embodiments, methods provided herein comprise an immunoassay to detect the presence of the antibody-anti-ACE2 IgM antibody complex. In some embodiments, the immunoassay is an ELISA assay.

In some embodiments, the subject is diagnosed as being infected with a SARS-CoV2 virus when the presence of the anti-ACE2 IgM antibody is detected. In some embodiments, the subject is suspected of being infected (e.g., not yet diagnosed) with the SARS-CoV2 virus when the presence of the anti-ACE2 IgM antibody is detected. In some embodiments, the presence of the anti-ACE2 IgM antibody is detected at the onset of severe disease in the subject.

As used herein, the term “control sample” or “reference sample” means a sample from a subject known not to have a rheumatic disease or immune disorder.

The term “comparing” as used herein encompasses comparing the level of the peptide or polypeptide comprised by the sample to be analyzed with a level of a suitable reference level specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample or a ratio of amounts is compared to a reference ratio of amounts. The comparison referred to in the methods of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format.

### Methods of Treatment for Severe COVID19 Symptoms

Disclosed herein are methods for treating a subject infected with a SARS-CoV2 virus or suspected of being infected with the SARS-CoV2 virus, the methods including (a) providing a biological sample from the subject; (b) detecting the presence of an anti-ACE2 IgM antibody in the biological sample, wherein the presence of the anti-ACE2 IgM antibody subject indicates the probability of severe COVID19 symptoms; and (c) administering to the subject a COVID19 treatment, thereby treating the subject infected with the SARS-CoV2 virus.

In some embodiments, a severe COVID19 symptom can include one of more of: high fever, severe cough, shortness of breath, difficulty breathing, fatigue, muscle aches, headache, loss of taste or smell, congestion, nausea, persistent pain or pressure in the chest, confusion, inability to wake or stay awake, delirium, seizures, or stroke.

Also disclosed herein are methods of screening for a clinically effective therapeutic agent to treat a subject infected with a SARS-CoV2 virus, the methods including (a) providing a biological sample from the subject; (b) detecting the presence of an anti-ACE2 IgM antibody in the biological sample; and (c) screening for a clinically effective therapeutic agent to administer to the subject as a COVID19 treatment.

In some embodiments, a COVID19 treatment can include an immunosuppressing agent, an anti-SARS-CoV2 monoclonal antibody, an antiviral agent, supplemental oxygen, or mechanical ventilatory support. In some embodiments, a COVID19 treatment can include an anti-SARS-CoV2 monoclonal antibody. In some embodiments, an anti-SARS-CoV2 monoclonal antibody can target a SARS-CoV2 spike protein. In some embodiments, an anti-SARS-CoV2 monoclonal antibody can include casirivimab. In some embodiments, an anti-SARS-CoV2 monoclonal antibody can include imdevimab. In some embodiments, an anti-SARS-CoV2 monoclonal antibody can include casirivimab and imdevimab. In some embodiments, an anti-SARS-CoV2 monoclonal antibody can include sotrovimab. In some embodiments, an anti-SARS-CoV2 monoclonal antibody can include bamlanivimab. In some embodiments, an anti-SARS-CoV2 monoclonal antibody can include etesevimab. In some embodiments, an anti-SARS-CoV2 monoclonal antibody can include bamlanivimab and etesevimab. In some embodiments, a COVID19 treatment can include an antiviral agent. In some embodiments, an antiviral agent can include molnupiravir.

In some embodiments, provided herein are methods for post-exposure prophylaxis or treatment of a SARS coronaviral infection in a subject in need thereof comprising detecting IgM antibodies directed against ACE2 in the subject and administering to the subject an effective amount of an agent that suppresses immune response. In some embodiments, an agent can be a corticosteroid. In other embodiments the agent can be an agent which suppresses Bruton's tyrosine kinase and/or IL-2-inducible T cell kinase in the immune cells of the subject.

As used herein, the term “tyrosine kinase inhibitor” refers to a family of small molecules or peptides with the ability to inhibit either cytosolic or receptor tyrosine kinases. Inhibition by this class of agents is through direct competition for ATP binding to the tyrosine kinase (e.g., genistein, lavendustin C, PP1-AG1872, PP2-AG1879, SU6656, CGP77675, PD166285, imatinib, erlotinib, gefitinib), allosteric inhibition of the tyrosine kinase (e.g., lavendustin A), inhibition of ligand binding to receptor tyrosine kinases (e.g., cetuximab), inhibition of tyrosine kinase interaction with other proteins (e.g., UCS15A, p60-v-Src inhibitor peptide) or destabilization of the tyrosine kinase (e.g., herbimycin A and radicicol).

Calcineurin is a calcium and calmodulin dependent serine/threonine protein phosphatase (also known as protein phosphatase 3, and calcium-dependent serine-threonine phosphatase). It activates the T cells of the immune system and can be blocked by drugs. Calcineurin activates nuclear factor of activated T cell cytoplasmic (NFATc), a transcription factor, by dephosphorylating it. The activated NFATc is then translocated into the nucleus, where it upregulates the expression of interleukin 2 (IL-2), which, in turn, stimulates the growth and differentiation of the T cell response. Calcineurin is the target of a class of drugs called calcineurin inhibitors, which include cyclosporin, voclosporin, pimecrolimus and tacrolimus. Therefore, in accordance with another embodiment, the agent that suppresses immune response is a calcineurin inhibitor.

### Kits and Devices

A “kit,” as used herein, typically includes a package or an assembly including one or more of the compositions or devices of the invention, and/or other compositions or devices associated with the invention, as previously described. Each of the compositions of the kit, if present, may be provided in liquid form (e.g., in solution), or in solid form (e.g., a dried powder). In certain embodiments, one or more of the compositions may be constitutable or otherwise processable (e.g., to an active form), for example, by the addition of a suitable solvent or other species, which may or may not be provided with the kit. A kit may further include other compositions or components associated with the invention include, but are not limited to, solvents, surfactants, diluents, salts, buffers, emulsifiers, chelating agents, fillers, antioxidants, binding agents, bulking agents, preservatives, drying agents, packaging materials, tubes, bottles, filters, containers, tapes, or adhesives. A kit may include instructions in any form that are provided in connection with the compositions of the invention in such a manner that one of ordinary skill in the art would recognize that the instructions are to be associated with the compositions of the invention. The instructions may be provided in any form recognizable by one of ordinary skill in the art as a suitable vehicle for containing such instructions. For example, written or published, verbal, audible (e.g., telephonic), digital, optical, visual (e.g., videotape, DVD, etc.) or electronic communications (including Internet or web-based communications), provided in any manner.

Disclosed herein are kits including (a) a lateral flow device comprising a first pad, a second pad, and a membrane, wherein the first pad receives a biological sample from a subject having a SARS-CoV2 infection, the second pad comprises an antibody that binds to an anti-ACE2 IgM antibody, and the membrane that reacts to the antibody bound to the anti-ACE2 IgM antibody; and (b) instructions to use the kit to detect the anti-ACE2 IgM antibody in the biological sample.

The term “device” as used herein relates to a system comprising the aforementioned units operatively linked to each other as to allow the diagnosis or monitoring according to the methods of the invention. Preferred detection agents which can be used for the analyzing unit are disclosed elsewhere herein. The analyzing unit, preferably, comprises the detection agents in immobilized form on a solid support which is to be contacted to the sample comprising the biomarkers the level of which is to be determined. Moreover, the analyzing unit can also comprise a detector which determines the level of binding ligand which is specifically bound to the biomarker(s). The determined level can be transmitted to the evaluation unit. The evaluation unit comprises a data processing element, such as a computer, with an implemented algorithm for carrying out a comparison between the determined level and a suitable reference (e.g. a reference level, or the level of the marker in a first or second sample from the individual). Suitable references can be derived from samples of individuals to be used for the generation of reference levels as described elsewhere herein above. The results may be given as output of parametric diagnostic raw data, preferably, as absolute or relative levels. It is to be understood that these data will need interpretation by the clinician. However, also envisaged are expert system devices wherein the output comprises processed diagnostic raw data the interpretation of which does not require a specialized clinician.

In some embodiments, the presently disclosed subject matter provides a lateral flow device adapted to perform the method disclosed herein for detecting an anti-ACE2-IgM antibody in a biological sample of a subject infected with a SARS-CoV2 virus.

In accordance with some embodiments, presently disclosed subject matter provides a dipstick assay to perform the method disclosed herein for detecting an anti-ACE2-IgM antibody in a biological sample of a subject infected with a SARS-CoV2 virus.

### Point-Of-Care Diagnostics such as Lateral Flow Devices and Dipstick Assays: Theory and Current Applications

The ability to provide test results rapidly to the patient and/or healthcare provider is very important to impact outcomes of multiple conditions. Rapid tests to aid diagnosis and enable early detection of multiple diseases and physiologic conditions are being developed. Such tests are especially useful when they can be applied with self-testing and require little in the way of laboratory processing. Examples of point-of-care (POC) test devices in common use today include pregnancy and fertility tests, as well as assays to follow blood glucose in diabetics. Development of diagnostic tests for infections that use POC testing are especially important in resource-poor settings; for this reason, POC testing has become a new goal to be achieved for infections such as HIV, malaria, and hepatitis. Similarly, POC testing has the potential of impacting clinical outcomes when applied to infections that occur in the outpatient setting, not only by providing indications of disease, but by enabling development of more robust prevention algorithms.

Commonly used immunoassays in diagnostic and research use include radio-immunoassays and enzyme-linked immunosorbent assays (ELISAs). Many of these elaborately configured immunoassays use monoclonal antibodies (mAbs) that possess the ability to bind specifically to the analyte being tested, thereby enhancing the accuracy of the assay. Various approaches have been described for carrying out enzyme immunoassays. A considerable number of these approaches, starting with the earliest of ELISAs, are solid-phase immunoassays in which the analyte to be detected is bound to a solid matrix directly (Direct ELISA) or indirectly (Sandwich ELISA), in which the analyte is captured on a primary reagent. The choice of the solid matrix depends on procedural considerations. A common matrix is the polystyrene surface of multi-well microtiter plates.

These types of assays also are amenable to developing POC devices, in which systems can be self-contained so that output is readable by the user. This characteristic is especially useful when collection of a sample to be tested does not require medical intervention (e.g., urine, saliva, or sputum). One device that enables this is the lateral-flow device (LFD). These devices use a multi-layered construction containing both absorbent and non-absorbent components to form a solid-phase. The capture and/or recognition reagents (antigen or antibody) are pre-applied to specific areas within the assembled apparatus and the analyte is allowed to flow through the system to come into contact with reagents. Often, for the purpose of self-containment, the reagent components are added in a dried state so that fluid from the sample re-hydrates and activates them. Conventional ELISA techniques can then be used to detect the analyte in the antigen-antibody complex. In some embodiments, the system can be designed to provide a colorimetric reading for visual estimation of a binary response (‘yes’ or ‘no’), or it can be configured to be quantitative.

Lateral flow devices are used to detect analytes in multiple body fluids, including serum and urine. To date, these types of devices have seen the most use for detecting circulating endogenous analytes; perhaps the most common use of this type of device is in the ubiquitous POC pregnancy test. Current efforts are being directed toward detecting microbial analytes, including nucleic acids, in the setting of viral infections (e.g., influenza, respiratory syncytial virus, and the like), Nielsen, K., et al., Prototype single step lateral flow technology for detection of avian influenza virus and chicken antibody to avian influenza virus. J Immunoassay Immunochem, 2007. 28(4): p. 307-18; Mokkapati, V. K., et al., Evaluation of UPlink-RSV: prototype rapid antigen test for detection of respiratory syncytial virus infection. Ann N Y Acad Sci, 2007. 1098: p. 476-85; bacterial infections (e.g., S. pneumoniae, Legionella, Mycobacteria), Koide, M., et al., Comparative evaluation of Duopath Legionella lateral flow assay against the conventional culture method using Legionella pneumophila and Legionella anisa strains. Jpn J Infect Dis, 2007. 60(4): p. 214-6.

A standard ELISA format was used as a screen to identify antibodies to use for capture on the immobilized device. The identified antibody can be used as a capture antibody with point of care testing device (strip), which can be optimized for conditions to detect galF-antigen (antibody concentration, incubation conditions, and the like).

The term “dipstick assay” as used herein means any assay using a dipstick in which sample solution is contacted with the dipstick to cause sample solution to move by capillary action to a capture zone of the dipstick thereby allowing a target antigen in the sample solution to be captured and detected at the capture zone. To test for the presence of analyte, the contact end of the dipstick is contacted with the test solution. If analyte is present in the test solution it travels to the capture zone of the dipstick by capillary action where it is captured by the capture antibody. The presence of analyte at the capture zone of the dipstick is detected by a further anti-analyte antibody (the detection antibody) labelled with, for example, colloidal gold.

These dipstick tests have several advantages. They are easy and cheap to perform, no specialist instruments are required, and the results are obtained rapidly and can be read visually. These tests are, therefore, particularly suited for use in a physician's office, at home, in remote areas, and in developing countries where specialist equipment may not be available. They can be used, for example, to test whether a patient has anti-ACE2-IgM antibodies.

To perform a method of the first aspect of the invention, the targeting agent and labels may simply be added to the test solution and the test solution then contacted with the contact end of the chromatographic strip. Such methods are easier to perform than the method disclosed in WO 00/25135 in which two separate wicking steps are required. The results may, therefore, be obtained more rapidly, and yet the sensitivity of analyte detection is higher.

The term “chromatographic strip” is used herein to mean any porous strip of material capable of transporting a solution by capillarity. The chromatographic strip may be capable of bibulous or non-bibulous lateral flow, but preferably bibulous lateral flow. By the term “non-bibulous lateral flow” is meant liquid flow in which all of the dissolved or dispersed components of the liquid are carried at substantially equal rates and with relatively unimpaired flow laterally through the membrane as opposed to preferential retention of one or more components as would occur with “bibulous lateral flow.” Materials capable of bibulous lateral flow include paper, nitrocellulose, and nylon. A preferred example is nitrocellulose.

The labels may be bound to the ligands of the targeting agent by pre-mixing the targeting agent with the labels before the targeting agent is added to (or otherwise contacted with) the test solution. However, in some circumstances, it is preferred that the targeting agent and labels are not pre-mixed because such pre-mixing can cause the targeting agent and labels to precipitate. Thus, the targeting agent and the labels may be added separately to (or contacted separately with) the test solution. The targeting agent and the labels can be added to (or contacted with) the test solution at substantially the same time, or in any order.

The test solution may be pre-incubated with the targeting agent and labels before the test solution is contacted with the contact end of the chromatographic strip to ensure complex formation. The optimal time of pre-incubation will depend on the ratio of the reagents and the flow rate of the chromatographic strip. In some cases, pre-incubation for too long can decrease the detection signal obtained, and even lead to false positive detection signals. Thus, it may be necessary to optimize the pre-incubation time for the particular conditions used.

It may be desired to pre-incubate the targeting agent with the test solution before binding the labels to the targeting agent so that the targeting agent can be allowed to bind to analyte in the test solution under optimum binding conditions.

In some embodiments, the indicator zone further comprises a test line and a control line. A test line can comprise an immobilized binding reagent. When antibodies are used to develop a test line in the LFD that employs a sandwich type of assay, they are applied at a ratio of about 1-3 μg/cm across the width of a strip 1 mm wide; hence, antibody concentration is about 10-30 μg/cm²; which is about 25-100 fold that used in an ELISA. Brown, M. C., Antibodies: key to a robust lateral flow immunoassay, in Lateral Flow Immunoassay, H.Y.T. R. C. Wong, Editor. 2009, Humana Press: New York, New York. p. 59-74.

For quality control, typically a lateral flow membrane can include a control zone comprising a control line. The term “control zone” refers to a portion of the test device comprising a binding molecule configured to capture the labeled reagent. In a lateral flow assay, the control zone may be in liquid flow contact with the detection zone of the carrier, such that the labeled reagent is captured on the control line as the liquid sample is transported out of the detection zone by capillary action. Detection of the labeled reagent on the control line confirms that the assay is functioning for its intended purpose. Placement of a control line can be accomplished using a microprocessor controlled TLC spotter, in which a dispenser pump releases a constant volume of reagent across the membrane.

A typical lateral flow device can also comprises an absorbent pad. The absorbent pad comprises an “absorbent material,” which as used herein, refers to a porous material having an absorbing capacity sufficient to absorb substantially all the liquids of the assay reagents and any wash solutions and, optionally, to initiate capillary action and draw the assay liquids through the test device. Suitable absorbent materials include, for example, nitrocellulose, nitrocellulose blends with polyester or cellulose, untreated paper, porous paper, rayon, glass fiber, acrylonitrile copolymer, plastic, glass, or nylon.

In some embodiments, a lateral flow membrane is bound to one or more substantially fluid-impervious sheets, one on either side, e.g., a bottom sheet and a complimentary top sheet with one or more windows defining an application zone and an indicator zone.

A typical lateral flow device also can include a housing. The term “housing” refers to any suitable enclosure for the presently disclosed lateral flow devices. Exemplary housings will be known to those skilled in the art. The housing can have, for example, a base portion and a lid portion. The lid portion can include a top wall and a substantially vertical side wall. A rim may project upwardly from the top wall and may further define a recess adapted to collect a sample from a subject. Suitable housings include those provided in U.S. Pat. No. 7,052,831 to Fletcher et al and those used in the BD Directigen™ EZ RSV lateral flow assay device.

One of ordinary skill in the art upon review of the presently disclosed subject matter would appreciate that the pre-treatment steps can be performed in any particular order, e.g., in some embodiments, the sample can be diluted or concentrated and then filtered, whereas in other embodiments, the sample can be filtered and then diluted or concentrated.

Generally, the presently disclosed subject matter provides a method for diagnosing the probability of severe COVID19 symptoms in a subject infected with SARS-CoV2 or suspected of being infected with SARS-CoV2 by detecting the presence of anti-ACE2-IgM antibodies in a biological sample of the mammalian subject, the method comprising: (a) contacting the sample of with at least one antibody specific for at least one anti-ACE2-IgM antibodies in an effective amount to produce a detectable amount of antibody—anti-ACE2-IgM antibody complex; and (b) detecting the presence of at least one antibody—anti-ACE2-IgM antibody complex, wherein the detection of the presence of at least one antibody—anti-ACE2-IgM antibody complex is diagnostic of a high probability of severe infection in a the subject.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In particular embodiments, the subject is a human adult suspected of having, having, or susceptible of having a microbial infection.

The presently disclosed methods can be used to diagnose, for the prognosis, or the monitoring of a disease state or condition. As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing,” refers to the determination of whether a sample specimen exhibits one or more characteristics of a condition or disease. The term “diagnosing” includes establishing the presence or absence of, for example, a target antigen or reagent bound targets, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease.

The term “prognosis,” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition.

As used herein, the term “risk” refers to a predictive process in which the probability of a particular outcome is assessed. The term “monitoring,” such as in “monitoring the course of a disease or condition,” refers to the ongoing diagnosis of samples obtained from a subject having or suspected of having a disease or condition. The term “marker” refers to a molecule, including an anti-ACE2-IgM antibody, that when detected in a sample is characteristic of or indicates the presence of severe COVID19 disease.

The presently disclosed methods can use a lateral flow device or dipstick assay comprising an immunochromatographic strip test that relies on a direct (double antibody sandwich) reaction. Without wishing to be bound to any one particular theory, this direct reaction scheme is best used when sampling for larger analytes that may have multiple antigenic sites. Different antibody combinations can be used, for example different antibodies can be included on the capture (detection) line, the control line, and included in the mobile phase of the assay, for

The invention also provides kits for diagnostic, prognostic or therapeutic applications. For diagnostic/prognostic applications, such kits may include any or all of the following: assay reagents, buffers, antibodies, or the like. Moreover, the kit may, preferably, comprise standards, reference samples and control samples. In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

### Patient Data and Serum Samples

The study cohort was defined as inpatients who had: 1) a confirmed diagnosis of COVID-19; 2) survival to death or discharge; and 3) remnant specimens in the Johns Hopkins COVID-19 Remnant Specimen Biorepository, an opportunity sample that includes 59% of Johns Hopkins Hospital COVID-19 patients and 66% of patients with length of stay >=3 days.

Diagnosis of COVID-19 was defined as detection of SARS-CoV-2 using any PCR test with an Emergency Use Authorization from the U.S. Food and Drug Administration. Selection and frequency of other laboratory testing were determined by treating physicians. The primary clinical data source was JH-CROWN, a Johns Hopkins Medicine COVID-19 registry that integrates all clinical data for COVID-19 patients, including demographics, medical history, comorbid conditions, symptoms, medications, laboratory results, medical images, and comprehensive bedside flowsheet data, including vital signs, respiratory events, and intravenous medication titration¹. Patient outcomes were defined by the World Health Organization (WHO) COVID-19 disease severity scale as described above. For this study we combined adjacent WHO classes, dividing the inpatient population into two groups according to maximum WHO severity: patients who did not require mechanical ventilation (WHO class 3-5); those who required mechanical ventilation with our without additional support, such as intravenous pressors, continuous renal replacement therapy (CRRT) and/or extracorporeal membrane oxygenation (ECMO) who survived (WHO classes 6-7) or died (WHO class 8). Serum samples were selected for timing within 24 hours of onset of the maximum WHO class; when multiple samples were available, the specimen closest to the WHO class onset was used. The initial analysis used a random sample of 12-20 unique patient specimens from each of the 4 classes meeting the criteria above (depending upon specimen availability for the clinical class). To determine biomarker trajectory, we analyzed an expanded cohort of patients who had 3-4 consecutive sera per patient across the course of their hospitalization. Patient selection was determined solely by specimen availability. Where available, additional serum for anti-ACE2 IgM-positive individuals was requested from the remnant biorepository. These studies were approved by the JHU Institutional Review Board (IRB 00251725, IRB 00256018, 00256547), with a waiver of consent because all specimens and clinical data were de-identified by the Core for Clinical Research Data Acquisition of the Johns Hopkins Institute for Clinical and Translational Research; the study team had no access to identifiable patient data.

Patient numbers per analysis are denoted in the figure legends.

Disease and healthy control sera—Three autoimmune disease control cohorts consisted of the following. (i) Sera from N=25 patients with SLE from the Johns Hopkins Lupus Cohort. (ii) Sera from N=13 patients diagnosed with systemic sclerosis after evaluation at the Johns Hopkins Scleroderma Center. (iii) N=15 patients with necrotizing myopathy defined by a positive anti-HMGCR antibody status evaluated at the Johns Hopkins Myositis Center. Serum from N=30 patients with influenza diagnosed using the Cepheid Xpert Xpress Flu/RSV assay in the Johns Hopkins Emergency departments or in-patient units, were studied. 11 were evaluated in the ED and discharged to outpatient care; an additional 19 patients were hospitalized, and required oxygen therapy or assisted ventilation⁵. Sera from N=30 adult healthy control individuals were also studied. Informed consent for these samples was obtained following protocols approved by the JHU Institutional Review Board (NA_00039294, NA_00039566, #NA00007454, IRB00066509, IRB00091667).

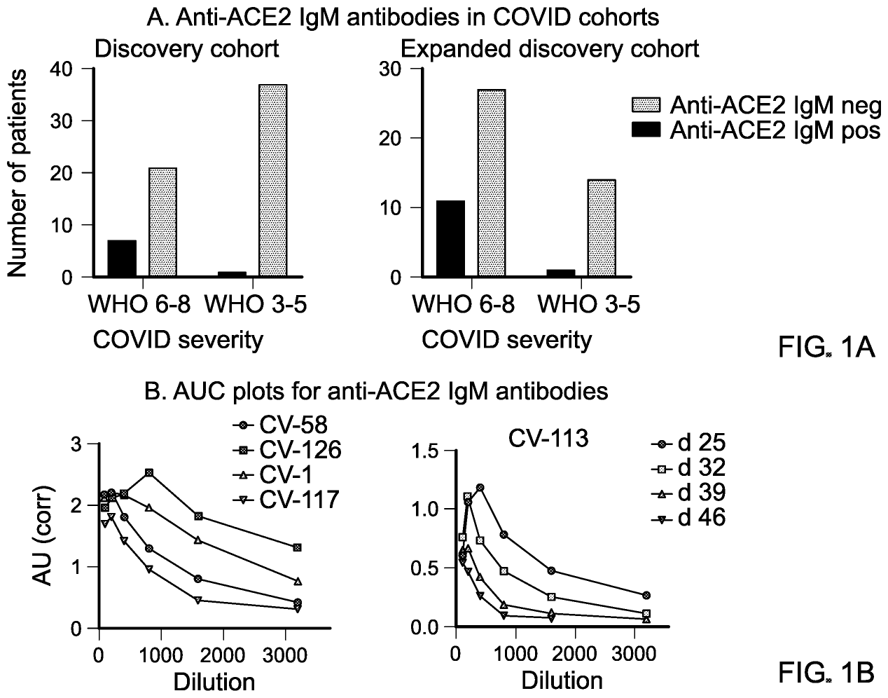

Anti-ACE2 and -SARSCoV2 spike ELISA assays—ELISA plate wells were coated overnight with 50 ng of purified protein (recombinant human ACE2 from Abcam, cat #ab151852: SARSCoV2 spike protein S1 subunit from Sino Bio cat #40591-V08B1) diluted in PBS. For each serum assayed, 2 wells were coated with protein (duplicate readout), and an adjacent well was incubated overnight with PBS only (to determine background specific to each sample tested). Anti-ACE2 IgM ELISA: Wells were washed with PBS plus 0.1% Tween (PBST), and subsequently blocked with 3% milk/PBST. Primary antibody incubations were routinely performed by diluting sera 1:200 in 1% milk/PBST overnight at 4° C. For the area under the curve plots (shown in FIG. 1B), serial serum dilutions ranging from 1:100 to 1:3,200 were used for the ELISA assays. Wells were then washed with PBST, followed by incubation with HRP-labeled anti-human IgM (Heavy chain-specific: Jackson ImmunoResearch cat #109-035-043) diluted 1:5000 in 1% milk/PBST (1 hour, room temperature). Color was developed with SureBlue peroxidase reagent (KPL). Reactions were terminated by adding HCl, and absorbances were read at 450 nM. The same anti-ACE2 IgM-positive reference serum was included on each plate assayed and all absorbances were calibrated relative to this reference serum. Anti-ACE2 IgG ELISA: was performed as described for anti-ACE2 IgM antibodies, with the following modifications. The concentration of Tween in PBST was 0.5%. Blocking was performed with 5% BSA/PBST, and sera and secondary antibodies were diluted with 1% BSA/PBST. The secondary antibody was HRP-labeled anti-human IgG (Jackson ImmunoResearch cat #109-036-088), diluted 1:10,000. The cutoff for assigning anti-ACE2 IgM and IgG antibody positivity was determined by assaying sera from 30 healthy controls. The mean ±3 SD of these values (0.340 and 0.187 calibrated OD units for anti-ACE2 IgM and IgG antibodies, respectively) was taken as the cutoff for each. The anti-ACE2 ELISA was validated by (i) blotting purified recombinant human ACE2 and (ii) using a second source of recombinant human ACE2 purchased from another vendor (Sino Biological, cat #10108-H08H). Anti-SARSCoV2 spike IgG ELISA—These assays were performed as described for anti-ACE2 IgG ELISAs, with the following modifications. Sera were assayed at a 1:1,200 dilution, and the primary antibody incubation was performed for 1 hr at room temperature.

All sera were obtained under Johns Hopkins IRB approved protocols. Healthy controls provided Informed Consent. Serum from patients with SARS-CoV-2 (COVID-19) was obtained with waiver of consent. Handling and disposal of all COVID biospecimens was performed according to requirements of the JHU Biosafety office.

At the initiation of this study, we were struck by the similar clinical presentation of severe COVID-19 to a dermatopulmonary syndrome, characterized by skin rash, rapidly progressive interstitial lung process with frequent progression to a need for ventilatory support, and a unique vasculopathic phenotype including cutaneous ulcers and digital ischemia.^{23,35} This syndrome has been associated with IgG autoantibodies to melanoma differentiation-associated 5 (MDA5). In its fulminant form, this can be viewed as a phenocopy of severe COVID-19, with a high mortality in the absence of treatment with steroids, IVIG, or calcineurin inhibition^{36,37}. Serum was available to us from a 42 year-old patient with this MDA5-associated syndrome, who developed symptoms of weakness, rash, fevers, and dyspnea in October of 2011. Her clinical course stabilized with immunosuppression consisting of corticosteroids, tacrolimus, and rituximab over the ensuing 8 years. Informed consent was obtained at presentation following protocols approved by the Johns Hopkins Institutional Review Board #NA00007454. Strikingly, this index patient had IgM and IgG autoantibodies against ACE2; her serum served as the reference calibrator on all ELISA plates. A study to understand the prevalence and relevance of these ACE2 autoantibodies in anti-MDA5-positive dermatomyositis-like disease and other rheumatic syndromes characterized by severe lung disease is currently ongoing.

CoronaChek assay—The CoronaChek serologic lateral flow assay (Hangzhou Biotest Biotech Co, Ltd., Hangzhou China) detects M (IgM) and G (IgG) antibodies to the spike protein and receptor binding domain of SARS-CoV-2. Studies on positive and negative control specimens from Maryland demonstrated: sensitivity of 95%, (95% CI 83%, 99%) in convalescent plasma donors an average of 50 days post symptom onset; sensitivity of 100% (95%CI 89%, 100%) in PCR confirmed hospitalized individuals 15 days after symptom onset; specificity of 100% 95% CI 94%, 100%) in pre pandemic patients infected with rhinoviruses and other coronaviruses.

Purification of IgM from patient serum—Following the manufacturer's instruction, 0.5 mL of POROS CaptureSelect™ IgM Affinity Matrix (Thermo Fisher Scientific) was equilibrated with 10 column volumes (CV) of phosphate-buffered saline (PBS) pH7.2 in a Poly-Prep® chromatography column (Bio-Rad). Patient serum samples (400 μL) were diluted 1:10 in PBS pH 7.2, filtered via centrifugation at 12,000×g using 0.45 μm spin filters (EMD Millipore), and loaded onto the column. The column was washed twice with 5 CV of PBS pH 7.2. Bound IgM protein was eluted with 5 CV of 0.1 M glycine, pH 3. The eluted IgM was immediately neutralized with 0.1 CV of 1 M Tris-HCl (pH 8).

The eluted IgM was exchanged into PBS and concentrated to match the original serum volume using Amicon 30 kDa molecular weight centrifugal filters (EMD Millipore). The 280 nm absorbance of the purified IgM was measured to calculate the IgM concentration, using the extinction coefficient for pentameric human IgM.

Biolayer interferometry analysis of ACE2/IgM interaction—Biolayer interferometry was performed using an Octet RED96 instrument (Molecular Devices) to measure the interaction of purified IgM from patient serum to ACE2. Wells of a black flat-bottom polypropylene plate (Corning) were loaded with the following samples: 50 nM biotinylated human ACE2 (Sino Biological, 10108-H08H-B); twofold dilutions of purified patient IgM; PBSA (PBS pH 7.2 containing 0.1% bovine serum albumin [BSA]); and regeneration buffer (0.1 M glycine, pH 3). All samples were centrifuged at 12,000×g through a 0.45 μm filter device (EMD Millipore), and buffers were vacuum filtered using a 0.22 μm membrane (EMD Millipore). ACE2 and the IgM samples were diluted in PBSA. ACE2 was loaded onto hydrated streptavidin (SA) biosensor tips (Molecular Devices), and baseline measurements were collected in PBSA. Binding kinetics were then measured by submerging the ACE2-coated biosensors in wells containing twofold serial dilutions of each patient IgM sample for 300 s (association) followed by submerging the biosensor in wells containing only PBSA for 450 s (dissociation). Tips were regenerated via exposure to regeneration buffer. Analysis and kinetic curve fitting (assuming a 1:1 binding model) was conducted using Octet Data Analysis HT software version 7.1 (Molecular Devices). Normalized equilibrium binding curves were obtained by plotting the response value after the 300 s association phase for each sample dilution and normalizing to the maximum value. Equilibrium curves were fitted to a single logistic model using a non-linear regression algorithm in GraphPad Prism software.

ACE2 activity assay—ACE2 activity was measured using a kit from BioVIsion (K897). Purified IgM (5 mg) or ACE2 inhibitor was preincubated with ACE2 in white Costar 96-well plates for 20 min at RT, followed by addition of fluorogenic ACE2 substrate as per the manufacturer's protocol. PBS made up 20% of the assay volume for CV-1 IgM and 10% for CV-64 IgM (due to lower protein concentration of the CV-1 IgM). Thus, a PBS control was included for each assay. The positive control contained only ACE2 and substrate, and the negative control was ACE2 plus ACE inhibitor and substrate. Fluorescence was measured every 5 min after substrate addition in a BMG Labtech FLUOstar Omega plate reader, with excitation at 355 nm and emission at 460 nm. Fluorescence values for wells containing no ACE2 (blank) were subtracted from the values shown.

Immunohistochemistry—Autopsies of 17 patients infected with Sars-CoV-2, documented by PCR on a pre or postmortem nasopharyngeal swab, were examined. Autopsies were consented for and performed on the clinical service with complete examination of chest organs and in-situ sampling of remaining organs and tissues, with histology performed on all sites. Lung paraffin sections from COVID-19 autopsy patients were either stained with Hematoxylin and Eosin, or processed as follows. After deparaffinization and rehydration, the sections were immersed in antigen retrieval solution (DAKO) for 30 min at 98° C. For IgM staining, the sections were blocked with goat serum (30 minutes at room temperature), followed by incubation with horseradish peroxidase labeled goat anti-human IgM (Jackson ImmunoResearch, cat # 109-035-043) diluted 1:500. Visualization was performed with a liquid DAB substrate-chromagen system (DAKO) and the sections were counterstained with hematoxylin before mounting.

Statistical Methods—The clinical measures used in this analysis are from the JHM Covid-19 Crown Registry that is actively curated by a team of clinicians, informaticists, and statisticians to assure data quality. For repeated measures outcomes (e.g. temperature, CRP, BMI) data was checked by making spaghetti plots' and visually checking the consistency of observations over time within an individual. The other main source are laboratory measurements of immune status (e.g. IgM or IgG antibodies) that are either binary indicators of presence/absence or absorbance levels as described in the immunoassay section.

To compare the rates of IgM antibody positivity between two subgroups, we estimated the ratio of the odds of positivity for one subgroup versus the other (odds ratio) and 95% confidence interval. Given the small numbers of patients in some comparator groups, we used a Fisher's exact test of the null hypothesis that the rates were equal (odds ratio=1). To compare means of continuous variables with roughly Gaussian distributions (determined using a quantile-quantile plot), we estimated the mean difference and its standard error and used an unpaired t-test of the null hypothesis that the two population means are equal. When we detected a large deviation from Gaussianity (for S protein IgG), a non-parametric test (Mann-Whitney) was used instead.

To compare the trajectory of clinical outcomes over time between IgM positive and IgM negative groups, we used a linear mixed effects model⁹. Variables were transformed to the log-scale if their marginal distribution was more nearly symmetric after transformation. The fixed effects included an indicator variable for IgM-positive status a smooth function of time (natural cubic spline with 3 degrees of freedom) and their interaction. We assumed each person had a random intercept and random linear trend to account for the likely correlation among repeated observations on individuals. Given this specification, we estimated the smooth curve for the IgM positive and negative groups as well as their difference with 95% confidence intervals. We tested the null hypothesis that the two population time curves are the same (coefficients for main effect of IgM and interaction of IgM with time all equal 0) using a Wald test statistic that was compared to a Chi-square distribution with 4 degrees of freedom. The analysis was repeated using natural splines with 2 to 4 degrees of freedom to assure that the findings were not sensitive to these assumptions.

### Example 1

The ELISA assays were then used to screen for IgM and IgG autoantibodies to ACE2, and applied these to a cohort of 66 hospitalized patients with COVID19 that reached the 5 most severe WHO ordinal categories as their maximal severity (28 severe, 38 moderate). Eight patients were positive for ACE2 IgM autoantibodies. Seven of these were in the ventilated (WHO 6/7) or dead groups (WHO 8) (7/28; 25%), while only a single patient was positive among the 38 patients who were not intubated (1/38; 2.6%; OR 12.3, 95% CI 1,875-141.9; p=0.0084; Fisher's exact test; FIG. 1A).

In order to increase sample size and define the stability and kinetics of these antibodies, we assembled additional patients in whom serum was available from multiple bleeds taken across their hospitalization. In order to increase sample size and define the stability and kinetics of these antibodies, we assembled additional patients in whom serum was available from multiple bleeds taken across their hospitalization. This added 52 COVID19 patients for analysis (38 in WHO ordinal groups 6-8 [31 ventilated and 7 dead], and 14 patients in ordinal category 4). The frequencies of anti-ACE2 IgM in these patients were very similar to the initial group: 11/38 (28.9%) of the patients with severe COVID were positive for anti-ACE2 IgM antibodies compared to 1/14 (7.1%) in the milder COVID group (FIG. 1B). The combined frequency of anti-ACE2 IgM in severe COVID was 18 of 66 patients (27.2%) compared to 2 of 52 patients with moderate COVID (3.8%; p=0.0009; OR 9.38, 95% CI 2.38-42.0; Fisher's exact test; FIG. 1A).

IgM levels were robust (FIG. 1A, center); all positives were confirmed and quantified by serial dilution (representative examples in FIG. 1B). Anti-ACE2 IgG were found in 12/66 (18%) patients with severe COVID, and 6/52 (11.3%) patients with moderate disease (p=0.44, Fisher's exact test). Only 4/18 severe patients with anti-ACE2 IgM antibodies were also IgG positive (FIG. 1A). ACE2 is therefore a prominent autoantibody target in patients with COVID, with IgM autoantibodies quite strikingly associated with severe disease.

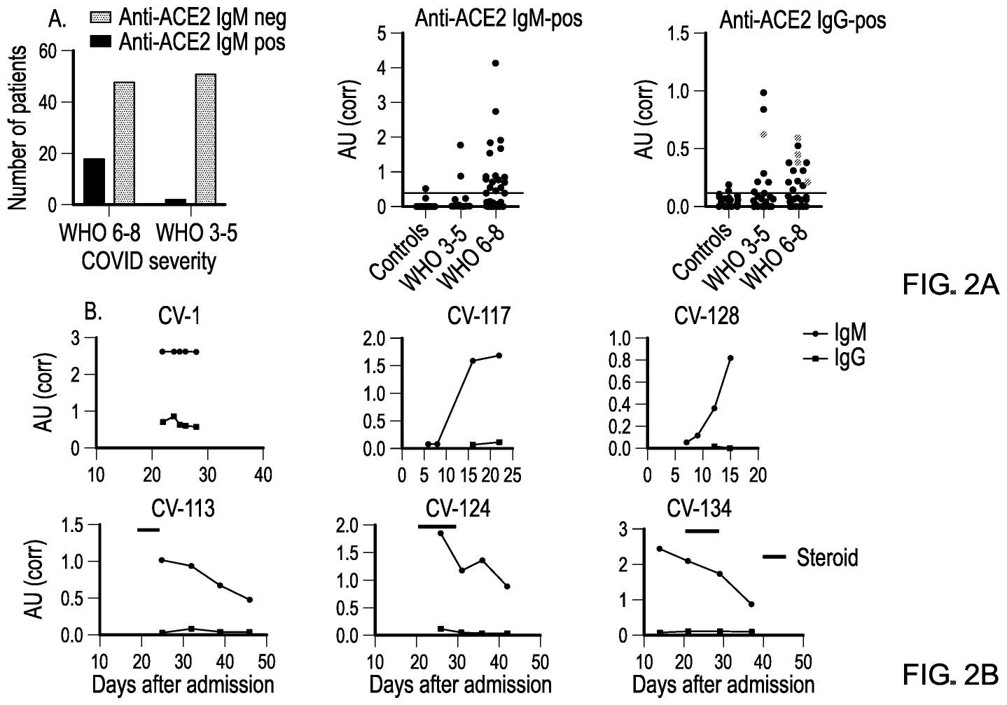

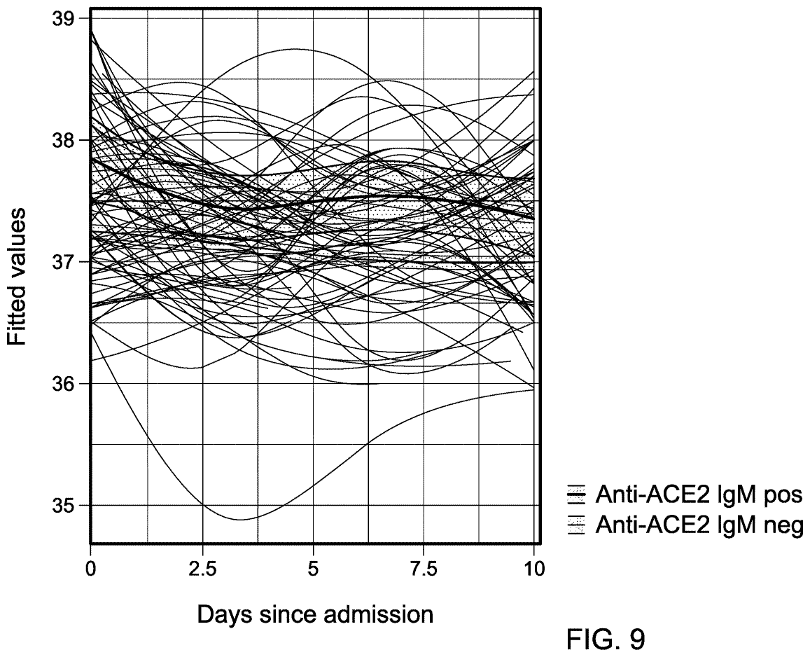

Clinical features of the anti-ACE2 IgM-positive group are summarized in FIG. 2 and Table 1 below. The mean age of the anti-ACE2 IgM-positive group was 61.5 years (N=20, se=9.7, S²=93.6), compared to 59.0 (N=98 se=17.3, S²=298.8) years for IgM-negatives (t=0.89, p=0.37, unpaired t-test). 72% of anti-ACE2 IgM were present in females. While the proportion of anti-ACE2 was higher in females (13/38, 34%) than males (5/28, 17.8%) with severe COVID-19, this difference did not reach statistical significance in this sample (p=0.17; Fisher's exact test). The mean BMI of IgM-positive patients was 35.4 (N=16, se=10.7, S²=115.2), compared to 30.4 (N=81, se=8.1, S²=65.2) in IgM-negative patients (t=1.74, p=0.10, unpaired t-test). Interestingly, the anti-ACE2-positive group had statistically significantly higher average temperatures over the first 10 days of hospitalization than the IgM-negative group (IgM-positive: mean=37.5, S²=0.65, N=783 on M=20 unique patients, IgM-negative: mean=37.0, S²=0.56, N=3137 on M=97 unique patients; chisq=22.72, p=0.0001 from linear mixed-effects model Wald test with 4 degrees of freedom (see statistical methods); FIG. 2D). The results do not qualitatively change when we restrict the analysis to the severe IgM-positive patients above and compare them to all severe COVID-19 patients from the CROWN Registry for whom IgM status were unknown (IgM-positive: mean=37.53, S²=0.64, N=721 on M=18 unique patients, IgM-unknown: mean=37.11, S²=0.59, N=14827 on M=473 unique patients; chisq=19.98, p=0.0005 from linear mixed-effects model Wald test with 4 degrees of freedom (see statistical methods), (FIG. 9). Population average CRP levels were also different in the 2 groups in the first 10 days after admission, with the population average peaking at ˜d4-d6 after admission at 20 mg/dL in the IgM-positive group, compared to 7.4 mg/dL for the IgM-negative group (IgM-positive: mean=16.96, S²=104.55, N 32 95 on M 32 18 unique patients, IgM-negative: mean=13.52, S²=151.58, N=413 on M=90 unique patients; chisq =11.19, p =0.02, from linear mixed-effects model Wald test with 4 degrees of freedom (see statistical methods), FIG. 2E).

TABLE 1
Demographics of the study population

| | N |
|---|---|
| Total | 118 |

Demographics

| | | |
|---|---|---|
| Age (years) | 60 | (50-71) |

| | | |
|---|---|---|
| Male gender | 56% | 66 |
| White race/ethnicity | 26% | 31 |
| Black race/ethnicity | 41% | 48 |
| Hispanic race/ethnicity | 23% | 27 |
| Asian race/ethnicity | 3% | 3 |
| Other | 8% | 9 |

| | | |
|---|---|---|
| BMI (kg/m2) | 30.2 | (26.0-34.9) |

Comorbidities

| | | |
|---|---|---|
| Diabetes mellitus | 47% | 56 |
| Hypertension | 64% | 76 |
| Coronary artery disease | 24% | 28 |
| Congestive heart failure | 23% | 27 |
| Chronic lung disease | 26% | 31 |

Maximum WHO class

| | | |
|---|---|---|
| Minimal oxygen | 28% | 34 |
| HFNC/NIPPV | 15% | 18 |
| Mechanical ventilation | 32% | 38 |
| Dead | 24% | 28 |

Continuous variables are median +/− interquartile range
Categorical variables are percentages

Various infectious and autoimmune disease controls were also tested for anti-ACE2 IgM (FIG. 2F). Anti-ACE2 IgM autoantibodies were not observed in 30 patients with acute influenza infection (including 11 patients evaluated in the ED, and 19 hospitalized patients for oxygen therapy or assisted ventilation⁵), 25 patients with SLE, 13 with scleroderma, and 15 with necrotizing myopathy. This specificity of anti-ACE2 IgM for severe COVID is striking.

### Example 2

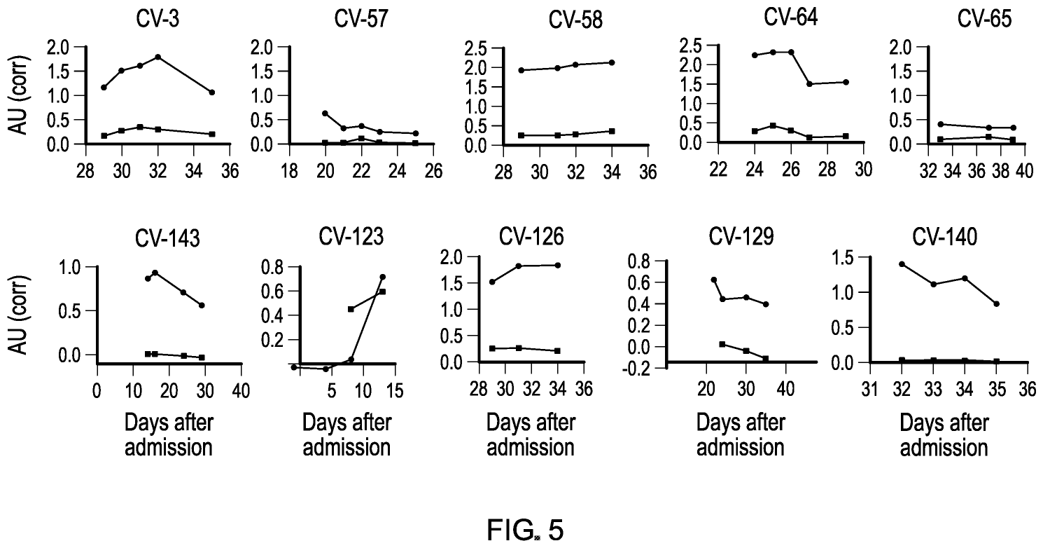

Since IgM is the earliest isotype elaborated in immune responses, we pursued a longitudinal analysis of anti-ACE2 IgM on all positive patients for whom serum was available. This demonstrated several patterns (FIG. 1B, and FIG. 5): (i) In 3 patients (CV-117, CV-123, CV-128), sampling spanned the development of anti-ACE2 IgM (data from 2 representative examples is shown in FIG. 1B). In these cases, autoantibodies appeared at ˜10 days after admission, and around the time of clinical worsening and intubation. We have not captured sufficient numbers of events around this time to make a definitive statement about onset of antibodies, but they do not appear to significantly precede clinical worsening; (ii) Anti-ACE2 IgM were already elevated at the first time point assayed in most patients, where patients were already intubated; in 4 patients (CV-1, CV-58, CV-65, CV-126), levels remained stable over time (one example shown in FIG. 1B; additional examples in FIG. 5); (iii) In a third group, anti-ACE2 IgM levels decreased over time (CV-113, CV-124 and CV-134 (FIG. 1B); CV-3, CV-57, CV-64, CV-129, CV-140, CV-143 (FIG. 5)).

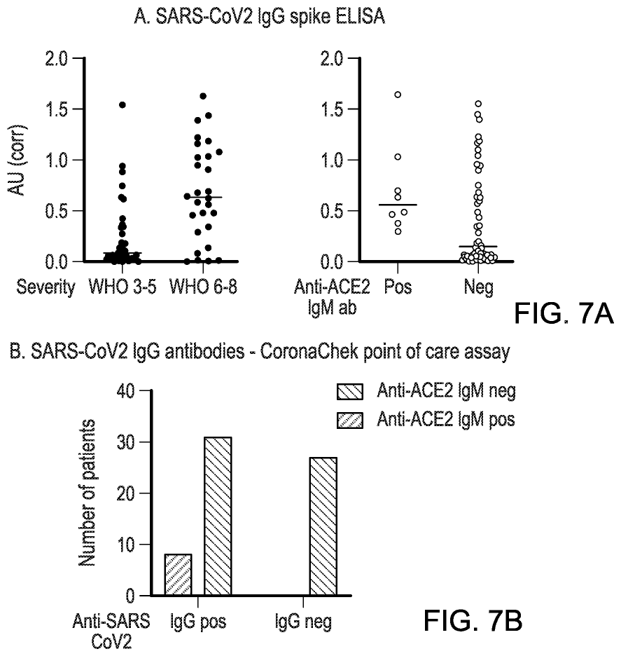

Overall, our studies captured multiple individuals where IgM levels waned over time (FIG. 1B, FIG. 5). In T cell-dependent immune responses, this generally occurs at the time of class switching to IgG. We therefore examined whether decreasing IgM levels over time were associated with increasing anti-ACE2 IgG levels at later time points. In one patient (CV-1), both IgG and IgM were present at the earliest point and remained constant over time (FIG. 1B). In 8 anti-ACE2 IgM-positive patients, we observed a decrease of anti-ACE2 IgM to ˜50% over time; all these patients were IgG-negative and remained so. Of note, these patients had robust IgG anti-SARS-CoV-2 Spike, demonstrating that the failure to undergo isotype switching was not a general feature in these patients. These data strongly suggest that the anti-ACE2 IgM immune response is not predominantly driven by T cells (either anti-viral or autoreactive), and may rather represent a T-independent antibody response induced by SARS-CoV2 infection. Such T-independent responses generally arise from B1 or marginal zone B cells,^{19, 20} and we suggest that such cells are the likely origin of this response. The finding of strikingly expanded circulating plasmablasts in severe COVID-19,^{21, 4, 22} a response which is oligoclonal with some clones that have not undergone somatic mutation, is consistent with this mechanism. An intriguing possibility is that a robust neutralizing anti-S IgG response induces an anti-idiotype IgM response, which also cross-reacts with ACE2, the S protein receptor. Multiple groups have noted that high levels of anti-S IgG occurring at the time of hospital admission are associated with more severe disease in COVID-19¹⁸. We assayed antibodies to SARS CoV2 S-protein by ELISA, and S- and NP antibodies by the CoronaChek point of care assay (FIG. 7). The mean ODs of anti-S antibodies were significantly higher in patients with severe compared to mild COVID (0.681 +/−0.478 vs 0.232 +/−0.334; mean +/−SD, P<0.0001; FIG. 7A). Anti-S IgG levels were also significantly increased in IgM-positive COVID patients compared to IgM-negatives (median vs 0.138; p=0.028; Mann-Whitney test; FIG. 7B). Using the CoronaChek assay, 8/8 (100%) of anti-ACE2 IgM-positive patients had a positive IgG result, compared to only 31/58 (53.4%) of anti-ACE2 IgM-negative patients (p=0.017, Fisher's exact test; FIG. 7C). Failure of the immune response to ACE2 to isotype switch to IgG is therefore not a general phenomenon in anti-ACE2 IgM patients, who have evidence of a robust anti-viral IgG response. The enhanced clinical inflammatory features (increased body temperature and CRP levels) that occur in anti-ACE2 IgM patients early after admission is of interest (FIGS. 2D & E), as it suggests that the amplification to severe disease may begin early in the disease course².

### Example 3

The clinical efficacy of steroids in patients with severe COVID¹³ prompted us to examine whether any of the anti-ACE2 IgM-positive patients had been treated with steroids, and whether there was any relationship to subsequent IgM levels. None of the 5 anti-ACE2 IgM-positive individuals who died were treated with steroids for more than 2 days. Of the 13 patients that were ventilated but survived, 6 were treated with steroids for more than 2 days, and there was a temporal association of decreased anti-ACE2 IgM levels with steroid treatment in 3 patients where appropriate samples were available (FIG. 1B). The data suggest that anti-ACE2 IgM might be a dynamic biomarker, and worthy of study in a prospective cohort where the effects of steroids can be rigorously addressed.

### Example 4

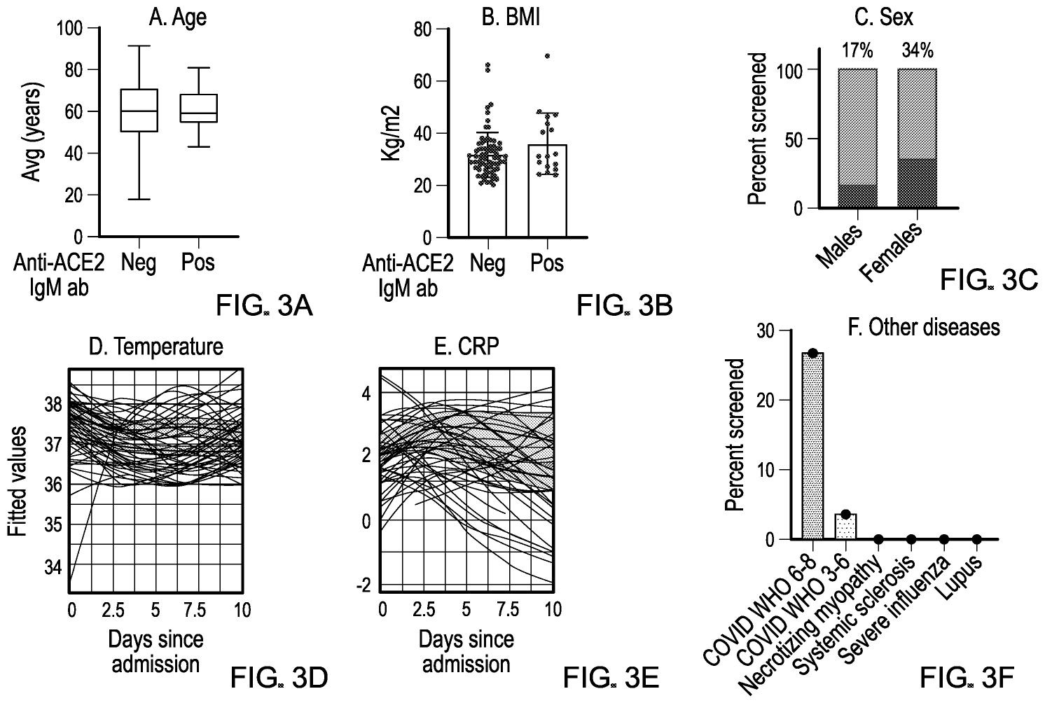

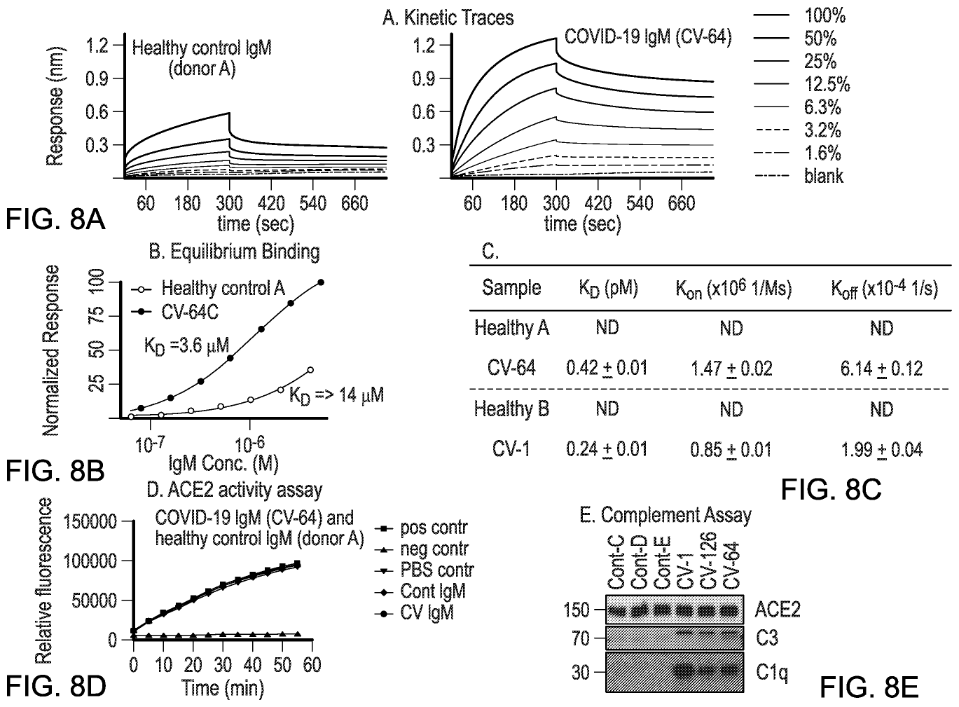

We next pursued additional analysis of the anti-ACE2 IgM binding properties using a different source of ACE2 antigen, and a different assay format. IgM purified from 2 patients with high titer ACE2 antibodies and 2 healthy controls were analyzed via biolayer interferometry. Patient IgM binding to immobilized ACE2 was saturable, with apparent KD values of 0.11 μM (CV-1) and 3.6 μM (CV-64), whereas IgM from healthy controls did not exhibit measurable binding to ACE2 (FIGS. 3A & B and FIG. 8, panels A-C). The reported KD values provide a ceiling for these measurements; since the purified IgM is a mixture of antibodies against various targets, the actual ACE2 affinities for individual IgM clones are presumably higher. These data are consistent with antibodies that have not undergone affinity maturation, which are known to have low affinity (high nanomolar to micromolar range) but benefit from avidity effects.²⁵

Since hypomorphic ACE2 function has been associated with severity in ARDS, we investigated whether purified IgM from COVID patients affected the catalytic function of ACE2 against a fluorogenic substrate. The purified IgM used above in binding assays had no effect on ACE2 activity (FIG. 3C and FIG. 8D).

IgM antibodies are mainly found in the circulation, where they are highly effective at activating the classical complement cascade at surfaces expressing their cognate antigens. 17 Purified IgM from patients CV-1, CV-64 and CV-126 and healthy controls were evaluated for their ability to activate the classical complement pathway after binding to plate-bound ACE2 (FIG. 3D).

IgM antibodies are mainly found in the circulation, where they are the most effective isotype at activating the classical complement cascade at surfaces expressing their cognate antigens.²⁶ We found that IgM antibodies with high affinity binding to ACE2 (i.e. CV-1) consistently activated complement upon antigen binding (FIG. 3D). In some experiments, IgM purified from CV-64 and CV-164 behaved similarly to CV-1, although the magnitude of the effect was decreased (FIG. 8E). These data suggest that IgM antibodies recognizing ACE2 play a role in the widespread complement pathway activation observed in COVID-19 patients,^{7,27}

### Example 5

Recent autopsy studies in COVID-19 have demonstrated a striking series of findings in the lung of COVID-19 patients.9,6 28 In addition to diffuse alveolar damage and perivascular infiltrating lymphocytes, there were striking angiocentric features in COVID-19 lungs, including severe endothelial injury associated with membrane disruption and ACE2 expression, widespread microangiopathy with occlusion of capillaries, and new vessel growth.

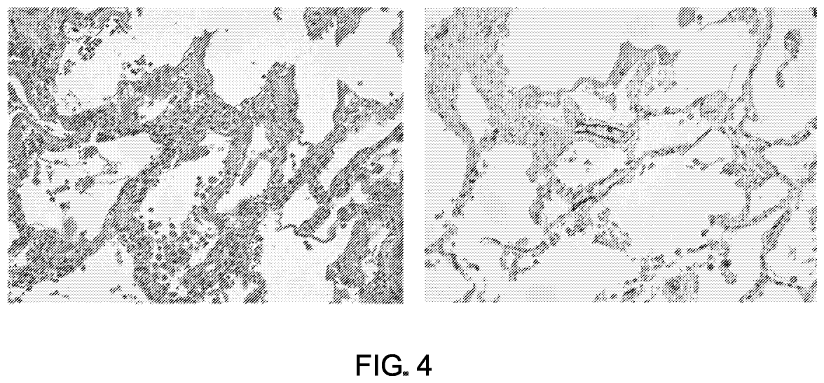

In order to define whether there was any in vivo evidence of IgM deposition in the lungs of patients with COVID-19, specimens from 17 autopsies were stained with anti-human IgM. Serum from the biorepository was only available in 4 of these patients; they were all negative for serum anti-ACE2 IgM, and had no IgM staining in the lung. Among the remaining 13 patients, 4 (30.7%) had evidence of staining with anti-IgM, with staining observed in blood vessels and capillaries in 2 patients (FIG. 4). Another patient had staining of a lymphatic channel, and the final patient had staining that was alveolar in pattern. Vascular endothelium appeared reactive, mostly without accompanying inflammatory infiltrates. The finding of examples of endothelial IgM deposition in COVID-19 lung demonstrates that an endothelial cell target with similar distribution to ACE2^{9,28} is recognized by IgM in a subgroup of COVID-19 patients with fatal disease. Defining whether these IgM molecules recognize ACE2 or another endothelial antigen is a high priority.

In order to define whether there was any in vivo evidence of IgM deposition in the lungs of patients with COVID, specimens from 17 autopsies were stained with anti-human IgM. Serum from the biorepository was only available in 4 patients; these were all negative for anti-ACE2 IgM, and had no IgM staining in the lung. Among the remaining 13 patients, 4 (30.7%) had evidence of staining with anti-IgM. In several patients, prominent staining in blood vessels and capillaries was observed (FIG. 4). This distribution is very similar to the staining described for ACE2. This finding of IgM staining in blood vessels and capillaries of the lung of a subgroup of patients who died after SARS-CoV-2 infection is of interest. Although we do not yet have direct evidence that this pathology represents the binding of anti-ACE2 IgM, it does demonstrate that an endothelial cell target with similar distribution to ACE2 is recognized by IgM in a subgroup of COVID patients with fatal disease. The finding of IgM deposition on endothelial cells in COVID is highly consistent with the striking endothelial injury and microthrombi that appear to be a signature pathological lesion in COVID.

Recent autopsy studies in COVID-19 have demonstrated a striking series of findings in the lung of COVID-19 patients.^{9, 28} In addition to diffuse alveolar damage and perivascular infiltrating lymphocytes, there were striking angiocentric features in COVID-19 lungs, including severe endothelial injury associated with membrane disruption and ACE2 expression, widespread microangiopathy with occlusion of capillaries, and new vessel growth.

In order to define whether there was any in vivo evidence of IgM deposition in the lungs of patients with COVID-19, specimens from 17 autopsies were stained with anti-human IgM. Serum from the biorepository was only available in 4 of these patients; they were all negative for serum anti-ACE2 IgM, and had no IgM staining in the lung. Among the remaining 13 patients, 4 (30.7%) had evidence of staining with anti-IgM, with staining observed in blood vessels and capillaries in 2 patients (FIG. 4). Another patient had staining of a lymphatic channel, and the final patient had staining that was alveolar in pattern. Vascular endothelium appeared reactive, mostly without accompanying inflammatory infiltrates. The finding of examples of endothelial IgM deposition in COVID-19 lung demonstrates that an endothelial cell target with similar distribution to ACE2^{9, 28} is recognized by IgM in a subgroup of COVID-19 patients with fatal disease. Defining whether these IgM molecules recognize ACE2 or another endothelial antigen is a high priority.

These studies demonstrate that anti-ACE2 IgM arise in the context of severe COVID-19, likely predominantly as a T-independent antibody response.¹⁹ This immune response is of potential pathogenic significance through binding to the surface of endothelial cells, activating the classical complement cascade, and initiating an inflammatory response. These mechanisms are potentially amenable to several readily available treatments, particularly short duration anti-inflammatory therapy (e.g. steroids and WIG therapy29, 30, 31 and potentially inhibitors of complement or therapies targeting T-independent antibody generation). It is noteworthy that mortality of the dermatopulmonary phenocopy associated with MDA5 autoantibodies appeared to be substantially decreased by steroids, IVIG and calcineurin inhibitor treatment,^{32, 33} although controlled trials have not been possible in this rare phenotype. Since ACE2 autoantibodies have features of T-independent responses, this may provide an important opportunity to use focused, short-term immune-focused therapies in severe COVID-19 (consistent with the dexamethasone results (13)) rather than deeper immunosuppression needed for T cell-driven processes.

In contrast to the recently described genetic and preexisting autoimmune factors that predispose to severe COVID-19 (i.e. inborn errors of type I IFN immunity and anti-IFN autoantibodies³⁴), this study adds a unique biomarker that results from SARS-CoV-2 infection and is strongly associated with severe clinical outcomes in patients with COVID-19. These distinct endophenotypes, driven by distinct mechanisms, having actionable markers and accounting for a substantial fraction of severe COVID-19, will likely benefit from both shared and distinct therapeutic approaches. Rapidly defining additional mechanistically-anchored groups in severe COVID is a high priority.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. P. Zhou, X.-L. Yang, X.-G. Wang, B. Hu, L. Zhang, W. Zhang, H.-R. Si, Y. Zhu, B. Li, C.-L. Huang, H.-D. Chen, J. Chen, Y. Luo, H. Guo, R.-D. Jiang, M.-Q. Liu, Y. Chen, X.-R. Shen, X. Wang, X.-S. Zheng, K. Zhao, Q.-J. Chen, F. Deng, L.-L. Liu, B. Yan, F.-X. Zhan, Y.-Y. Wang, G.-F. Xiao, Z.-L. Shi, A pneumonia outbreak associated with a new coronavirus of probable bat origin. Nature. 579, 270-273 (2020).
2. B. T. Garibaldi, J. Fiksel, J. Muschelli, M. L. Robinson, M. Rouhizadeh, J. Perin, G. Schumock, P. Nagy, J. H. Gray, H. Malapati, M. Ghobadi-Krueger, T. M. Niessen, B. S. Kim, P. M. Hill, M. S. Ahmed, E. D. Dobkin, R. Blanding, J. Abele, B. Woods, K. Harkness, D. R. Thiemann, M. G. Bowring, A. B. Shah, M.-C. Wang, K. Bandeen-Roche, A. Rosen, S. L. Zeger, A. Gupta, Patient Trajectories Among Persons Hospitalized for COVID-19: A Cohort Study. Annals of Internal Medicine (2020), doi:10.7326/M20-3905.
3. S. Richardson, J. S. Hirsch, M. Narasimhan, J. M. Crawford, T. McGinn, K. W. Davidson, and the N. C.-19 R. Consortium, D. P. Barnaby, L. B. Becker, J. D. Chelico, S. L. Cohen, J. Cookingham, K. Coppa, M. A. Diefenbach, A. J. Dominello, J. Duer-Hefele, L. Falzon, J. Gitlin, N. Hajizadeh, T. G. Harvin, D. A. Hirschwerk, E. J. Kim, Z. M. Kozel, L. M. Marrast, J. N. Mogavero, G. A. Osorio, M. Qiu, T. P. Zanos, Presenting Characteristics, Comorbidities, and Outcomes Among 5700 Patients Hospitalized With COVID-19 in the New York City Area. JAMA. 323, 2052-2059 (2020).
4. L. Kuri-Cervantes, M. B. Pampena, W. Meng, A. M. Rosenfeld, C. A. G. Ittner, A. R. Weisman, R. S. Agyekum, D. Mathew, A. E. Baxter, L. A. Vella, O. Kuthuru, S. A. Apostolidis, L. Bershaw, J. Dougherty, A. R. Greenplate, A. Pattekar, J. Kim, N. Han, S. Gouma, M. E. Weirick, C. P. Arevalo, M. J. Bolton, E. C. Goodwin, E. M. Anderson, S. E. Hensley, T. K. Jones, N. S. Mangalmurti, E. T. L. Prak, E. J. Wherry, N. J. Meyer, M. R. Betts, Comprehensive mapping of immune perturbations associated with severe COVID-19. Science Immunology. 5 (2020), doi:10.1126/sciimmunol.abd7114.
5. C. Lucas, P. Wong, J. Klein, T. B. R. Castro, J. Silva, M. Sundaram, M. K. Ellingson, T. Mao, J. E. Oh, B. Israelow, T. Takahashi, M. Tokuyama, P. Lu, A. Venkataraman, A. Park, S. Mohanty, H. Wang, A. L. Wyllie, C. B. F. Vogels, R. Earnest, S. Lapidus, I. M. Ott, A. J. Moore, M. C. Muenker, J. B. Fournier, M. Campbell, C. D. Odio, A. Casanovas-Massana, R. Herbst, A. C. Shaw, R. Medzhitov, W. L. Schulz, N. D. Grubaugh, C. D. Cruz, S. Farhadian, A. I. Ko, S. B. Omer, A. Iwasaki, Longitudinal analyses reveal immunological misfiring in severe COVID-19. Nature. 584, 463-469 (2020).
6. P. Domingo, I. Mur, V. Pomar, H. Corominas, J. Casademont, N. de Benito, The four horsemen of a viral Apocalypse: The pathogenesis of SARS-CoV-2 infection (COVID-19). EBioMedicine. 58 (2020), doi:10.1016/j.ebiom.2020.102887.
7. C. Magro, J. J. Mulvey, D. Berlin, G. Nuovo, S. Salvatore, J. Harp, A. Baxter-Stoltzfus, J. Laurence, Complement associated microvascular injury and thrombosis in the pathogenesis of severe COVID-19 infection: A report of five cases. Translational Research. 220, 1-13 (2020).
8. L.-A. Teuwen, V. Geldhof, A. Pasut, P. Carmeliet, COVID-19: the vasculature unleashed. Nature Reviews Immunology. 20, 389-391 (2020).
9. C. Bryce, Z. Grimes, E. Pujadas, S. Ahuja, M. B. Beasley, R. Albrecht, T. Hernandez, A. Stock, Z. Zhao, M. A. Rasheed, J. Chen, L. Li, D. Wang, A. Corben, K. Haines, W. Westra, M. Umphlett, R. E. Gordon, J. Reidy, B. Petersen, F. Salem, M. Fiel, S. M. E. Jamal, N. M. Tsankova, J. Houldsworth, Z. Mussa, W.-C. Liu, B. Veremis, E. Sordillo, M. Gitman, M. Nowak, R. Brody, N. Harpaz, M. Merad, S. Gnjatic, R. Donnelly, P. Seigler, C. Keys, J. Cameron, I. Moultrie, K.-L. Washington, J. Treatman, R. Sebra, J. Jhang, A. Firpo, J. Lednicky, A. Paniz-Mondolfi, C. Cordon-Cardo, M. Fowkes, medRxiv, in press, doi:10,1101/2020.05.18.20099960.
10. Large-Vessel Stroke as a Presenting Feature of Covid-19 in the Young|NEJM, (available at https://www.nejm.org/doi/full/10.1056/NEJMc2009787).
11. E. Driggin, M. V. Madhavan, B. Bikdeli, T. Chuich, J. Laracy, G. Biondi-Zoccai, T. S. Brown, C. D. Nigoghossian, D. A. Zidar, J. Haythe, D. Brodie, J. A. Beckman, A. J. Kirtane, G. W. Stone, H. M. Krumholz, S. A. Parikh, Cardiovascular Considerations for Patients, Health Care Workers, and Health Systems During the COVID-19 Pandemic. J Am Coll Cardiol. 75, 2352-2371 (2020).
12, L. M. Lam, S. J. Murphy, L. Kuri-Cervantes, A. R. Weisman, C. A. G. Ittner, J. P. Reilly, M. B. Pampena, M. R. Betts, E. J. Wherry, W.-C. Song, J. D. Lambris, D. B. Cines, N. J. Meyer, N. S. Mangalmurti, medRxiv, in press, doi:10. 1101/2020.05.20. 20104398.
13. RECOVERY Collaborative Group, Horby P, Lim W S, Emberson J R, Mafham M, Bell J L, Linsell L, Staplin N, Brightling C, Ustianowski A, Elmahi E, Prudon B, Green C, Felton T, Chadwick D, Rege K, Fegan C, Chappell L C, Faust S N, Jaki T, Jeffery K, Montgomery A, Rowan K, Juszczak E, Baillie J K, Haynes R, Landray M J. Dexamethasone in Hospitalized Patients with Covid-19—Preliminary Report. N Engl J Med. 2020 Jul. 17:NEJMoa2021436. doi: 10.1056/NEJMoa2021436. Epub ahead of print. PMID: 32678530; PMCID: PMC7383595.
14. J. Shang, G. Ye, K. Shi, Y. Wan, C. Luo, H. Aihara, Q. Geng, A. Auerbach, F. Li, Structural basis of receptor recognition by SARS-CoV-2. Nature. 581, 221-224 (2020).
15. I. Hamming, W. Timens, M. L. C. Bulthuis, A. T. Lely, G. J. Navis, H. van Goor, Tissue distribution of ACE2 protein, the functional receptor for SARS coronavirus. A first step in understanding SARS pathogenesis. The Journal of Pathology. 203, 631-637 (2004).
16. Y. Imai, K. Kuba, S. Rao, Y. Huan, F. Guo, B. Guan, P. Yang, R. Sarao, T. Wada, H. Leong-Poi, M. A. Crackower, A. Fukamizu, C.-C. Hui, L. Hein, S. Uhlig, A. S. Slutsky, C. Jiang, J. M. Penninger, Angiotensin-converting enzyme 2 protects from severe acute lung failure. Nature. 436, 112-116 (2005).
17. N. F. Chaisson, J. Paik, A.-M. Orbai, L. Casciola-Rosen, D. Fiorentino, S. Danoff, A. Rosen, A novel dermato-pulmonary syndrome associated with MDA-5 antibodies: report of 2 cases and review of the literature. Medicine. 91, 220-228 (2012).
18. K. L. Lynch, J. D. Whitman, N. P. Lacanienta, E. W. Beckerdite, S. A. Kastner, B. R. Shy, G. M. Goldgof, A. G. Levine, S. P. Bapat, S. L. Stramer, J. H. Esensten, A. W. Hightower, C. Bern, A. H. B. Wu, Magnitude and kinetics of anti-SARS-CoV-2 antibody responses and their relationship to disease severity. Clin. Infect. Dis. (2020), doi:10.1093/cid/ciaa979.
19. S. Fagarasan, T. Honjo, T-Independent immune response: new aspects of B cell biology. Science. 290, 89-92 (2000).
20. A. Cerutti, M. Cols, I. Puga, Marginal zone B cells: virtues of innatelike antibody-producing lymphocytes. Nat Rev Immunol. 13, 118-132 (2013).
21. S. De Biasi, D. Lo Tartaro, M. Meschiari, L. Gibellini, C. Bellinazzi, R. Borella, L. Fidanza, M. Mattioli, A. Paolini, L. Gozzi, D. Jaacoub, M. Faltoni, S. Volpi, J. Milié, M. Sita, M. Sarti, C. Pucillo, M. Girardis, G. Guaraldi, C. Mussini, A. Cossarizza, Expansion of plasmablasts and loss of memory B cells in peripheral blood from COVID-19 patients with pneumonia. Eur. J. Immunol. 50, 1283-1294 (2020).
22. A. J. Wilk, A. Rustagi, N. Q. Zhao, J. Roque, G. J. Martinez-Colon, J. L. McKechnie, G. T. Ivison, T. Ranganath, R. Vergara, T. Hollis, L. J. Simpson, P. Grant, A. Subramanian, A. J. Rogers, C. A. Blish, A single-cell atlas of the peripheral immune response in patients with severe COVID-19. Nature Medicine. 26, 1070-1076 (2020).
23. S. Sato, K. Hoshino, T. Satoh, T. Fujita, Y. Kawakami, T. Fujita, M. Kuwana, RNA helicase encoded by melanoma differentiation-associated gene 5 is a major autoantigen in patients with clinically amyopathic dermatomyositis: Association with rapidly progressive interstitial lung disease. Arthritis and Rheumatism. 60, 2193-2200 (2009).
24. T. W. R. E. A. for C.-19 T. (REACT) W. Group, J. A. C. Sterne, S. Murthy, J. V. Diaz, A. S. Slutsky, J. Villar, D. C. Angus, D. Annane, L. C. P. Azevedo, O. Berwanger, A. B. Cavalcanti, P.-F. Dequin, B. Du, J. Emberson, D. Fisher, B. Giraudeau, A. C. Gordon, A. Granholm, C. Green, R. Haynes, N. Heming, J. P. T. Higgins, P. Horby, P. Jüni, M. J. Landray, A. L. Gouge, M. Leclerc, W. S. Lim, F. R. Machado, C. McArthur, F. Meziani, M. H. Moller, A. Perner, M. W. Petersen, J. Savović, B. Tomazini, V. C. Veiga, S. Webb, J. C. Marshall, Association Between Administration of Systemic Corticosteroids and Mortality Among Critically Ill Patients With COVID-19: A Meta-analysis. JAMA (2020), doi:10.1001/jama.2020.17023.
25. H. N. Eisen, Affinity Enhancement of Antibodies: How Low-Affinity Antibodies Produced Early in Immune Responses Are Followed by High-Affinity Antibodies Later and in Memory B-Cell Responses. Cancer Immunol Res. 2, 381-392 (2014).
26. J. S. Brown, T. Hussell, S. M. Gilliland, D. W. Holden, J. C. Paton, M. R. Ehrenstein, M. J. Walport, M. Botto, The classical pathway is the dominant complement pathway required for innate immunity to Streptococcus pneumoniae infection in mice. PNAS. 99, 16969-16974 (2002).
27. J. C. Holter, S. E. Pischke, E. de Boer, A. Lind, S. Jenum, A. R. Holten, K. Tonby, A. Barratt-Due, M. Sokolova, C. Schjalm, V. Chaban, A. Kolderup, T. Tran, T. T. Gjolberg, L. G. Skeie, L. Hesstvedt, V. OrmAsen, B. Fevang, C. Austad, K. E. Muller, C. Fladeby, M. Holberg-Petersen, B. Halvorsen, F. Muller, P. Aukrust, S. Dudman, T. Ueland, J. T. Andersen, F. Lund-Johansen, L. Heggelund, A. M. Dyrhol-Riise, T. E. Mollnes, Systemic complement activation is associated with respiratory failure in COVID-19 hospitalized patients, 8.
28. M. Ackermann, S. E. Verleden, M. Kuehnel, A. Haverich, T. Welte, F. Laenger, A. Vanstapel, C. Werlein, H. Stark, A. Tzankov, W. W. Li, V. W. Li, S. J. Mentzer, D. Jonigk, Pulmonary Vascular Endothelialitis, Thrombosis, and Angiogenesis in Covid-19. New England Journal of Medicine. 383, 120-128 (2020).
29. M. Basta, M. C. Dalakas, High-dose intravenous immunoglobulin exerts its beneficial effect in patients with dermatomyositis by blocking endomysial deposition of activated complement fragments. The Journal of clinical investigation. 94, 1729-1735 (1994).
30. L. Mouthon, S. V. Kaveri, S. H. Spalter, S. Lacroix-Desmazes, C. Lefranc, R. Desai, M. D. Kazatchkine, Mechanisms of action of intravenous immune globulin in immune-mediated diseases. Clinical & Experimental Immunology. 104, 3-9 (1996).
31. F. Nimmerjahn, J. V. Ravetch, Anti-Inflammatory Actions of Intravenous Immunoglobulin. Annual Review of Immunology. 26, 513-533 (2008).
32. Koguchi-Yoshioka H, Okiyama N, Iwamoto K, Matsumura Y, Ogawa T, Inoue S, Watanabe R, Fujimoto M. Intravenous immunoglobulin contributes to the control of antimelanoma differentiation-associated protein 5 antibody-associated dermatomyositis with palmar violaceous macules/papules. Br J Dermatol. 2017 November;177(5):1442-1446. doi: 10.1111/bjd.15499. Epub 2017 Oct. 11. Erratum in: Br J Dermatol. 2020 May;182(5):1314. PMID: 28346662.
33. F. Romero-Bueno, P. Diaz Del Campo, E. Trallero-Araguas, J. C. Ruiz-Rodriguez, I. Castellvi, M. J. Rodriguez-Nieto, M. J. Martinez-Becerra, O. Sanchez-Pernaute, I. Pinal-Fernandez, X. Solanich, T. Gono, M. A. Gonzalez-Gay, M. N. Plana, A. Selva-O'Callaghan, MEDRA5 (Spanish MDA5 Register) group (listed contributors at the end of the article), Recommendations for the treatment of anti-melanoma differentiation-associated gene 5-positive dermatomyositis-associated rapidly progressive interstitial lung disease. Seminars in Arthritis and Rheumatism. 776-790 (2020).
34. P. Bastard, L. B. Rosen, Q. Zhang, E. Michailidis, H.-H. Hoffmann, Y. Zhang, K. Dorgham, Q. Philippot, J. Rosain, V. Béziat, J. Manry, E. Shaw, L. Haljasmagi, P. Peterson, L. Lorenzo, L. Bizien, S. Trouillet-Assant, K. Dobbs, A. A. de Jesus, A. Belot, A. Kallaste, E. Catherinot, Y. Tandjaoui-Lambiotte, J. L. Pen, G. Kerner, B. Bigio, Y. Seeleuthner, R. Yang, A. Bolze, A. N. Spaan, O. M. Delmonte, M. S. Abers, A. Aiuti, G. Casari, V. Lampasona, L. Piemonti, F. Ciceri, K. Bilguvar, R. P. Lifton, M. Vasse, D. M. Smadja, M. Migaud, J. Hadjadj, B. Terrier, D. Duffy, L. Quintana-Murci, D. van de Beek, L. Roussel, D. C. Vinh, S. G. Tangye, F. Haerynck, D. Dalmau, J. Martinez-Picado, P. Brodin, M. C. Nussenzweig, S. Boisson-Dupuis, C. Rodriguez-Gallego, G. Vogt, T. H. Mogensen, A. J. Oler, J. Gu, P. D. Burbelo, J. Cohen, A. Biondi, L. R. Bettini, M. D'Angio, P. Bonfanti, P. Rossignol, J. Mayaux, F. Rieux-Laucat, E. S. Husebye, F. Fusco, M. V. Ursini, L. Imberti, A. Sottini, S. Paghera, E. Quiros-Roldan, C. Rossi, R. Castagnoli, D. Montagna, A. Licari, G. L. Marseglia, X. Duval, J. Ghosn, H. Lab§, N.-U. I. R. to C. Group§, C. Clinicians§, C.-S. Clinicians§, I. C. Group§, F. C. C. S. Group§, T. M. I. Consortium§, C.-C. Cohort§, A. U. C.-19 Biobank§, C. H. G. Effort§, J. S. Tsang, R. Goldbach-Mansky, K. Kisand, M. S. Lionakis, A. Puel, S.-Y. Zhang, S. M. Holland, G. Gorochov, E. Jouanguy, C. M. Rice, A. Cobat, L. D. Notarangelo, L. Abel, H. C. Su, J.-L. Casanova, Auto-antibodies against type I IFNs in patients with life-threatening COVID-19. Science (2020), doi:10.1126/science.abd4585.
35. D. Fiorentino, L. Chung, J. Zwerner, A. Rosen, L. Casciola-Rosen, The mucocutaneous and systemic phenotype of dermatomyositis patients with antibodies to MDAS (CADM-140): A retrospective study. Journal of the American Academy of Dermatology. 65, 25-34 (2011).
36. H. Koguchi-Yoshioka, N. Okiyama, K. Iwamoto, Y. Matsumura, T. Ogawa, S. Inoue, R. Watanabe, M. Fujimoto, Intravenous immunoglobulin contributes to the control of antimelanoma differentiation-associated protein 5 antibody-associated dermatomyositis with palmar violaceous macules/papules. The British Journal of Dermatology. 177, 1442-1446 (2017).
37. F. Romero-Bueno, P. Diaz Del Campo, E. Trallero-Araguas, J. C. Ruiz-Rodriguez, I. Castellvi, M. J. Rodriguez-Nieto, M. J. Martinez-Becerra, O. Sanchez-Pernaute, I. Pinal-Fernandez, X. Solanich, T. Gono, M. A. Gonzalez-Gay, M. N. Plana, A. Selva-O'Callaghan, MEDRA5 (Spanish MDA5 Register) group (listed contributors at the end of the article), Recommendations for the treatment of anti-melanoma differentiation-associated gene 5-positive dermatomyositis-associated rapidly progressive interstitial lung disease. Seminars in Arthritis and Rheumatism. 776-790 (2020).

1. A method for diagnosing the probability of extended duration of severe COVID19 symptoms in a subject infected with a SARS-CoV2 virus or suspected of being infected with the SARS-CoV2 virus, the method comprising:
(a) providing a biological sample from the subject; and
(b) detecting the presence of an anti-ACE2 IgM antibody in the biological sample, thereby diagnosing the probability of extended duration of severe COVID19 symptoms in the subject.

2. The method of claim 1, wherein the extended duration of severe COVID19 symptoms is about 44 days.

3. A method for diagnosing the probability of severe COVID19 symptoms in a subject infected with a SARS-CoV2 virus or suspected of being infected with the SARS-CoV2 virus, the method comprising:
(a) contacting a biological sample from the subject with an antibody that binds to an anti-ACE2 IgM antibody in the biological sample, producing an antibody-anti-ACE2 IgM antibody complex; and
(b) detecting the presence of the antibody-anti-ACE2 IgM antibody complex, thereby diagnosing the probability of severe COVID19 symptoms in the subject.

4. The method of claim 3, wherein the method comprises an immunoassay to detect the presence of the antibody-anti-ACE2 IgM antibody complex.

5. The method of claim 4, wherein the immunoassay is an ELISA assay.

6. A method for treating a subject infected with a SARS-CoV2 virus or suspected of being infected with the SARS-CoV2 virus, the method comprising:
(a) providing a biological sample from the subject;
(b) detecting the presence of an anti-ACE2 IgM antibody in the biological sample, wherein the presence of the anti-ACE2 IgM antibody subject indicates the probability of severe COVID19 symptoms; and
(c) administering to the subject a COVID19 treatment, thereby treating the subject infected with the SARS-CoV2 virus.

7. The method of claim 1, wherein a severe COVID19 symptom comprises one of more of: high fever, severe cough, shortness of breath, difficulty breathing, fatigue, muscle aches, headache, loss of taste or smell, congestion, nausea, persistent pain or pressure in the chest, confusion, inability to wake or stay awake, delirium, seizures, or stroke.

8. A method of screening for a clinically effective therapeutic agent to treat a subject infected with a SARS-CoV2 virus, the method comprising:
(a) providing a biological sample from the subject;
(b) detecting the presence of an anti-ACE2 IgM antibody in the biological sample; and
(c) screening for a clinically effective therapeutic agent to administer to the subject as a COVID19 treatment.

9. The method of claim 8, wherein the COVID19 treatment comprises an immunosuppressing agent, an anti-SARS-CoV2 monoclonal antibody, an antiviral agent, supplemental oxygen, or mechanical ventilatory support.

10. The method of claim 1, wherein the biological sample is a blood sample.

11. The method of claim 1, wherein the presence of the anti-ACE2 IgM antibody is detected at the onset of severe disease in the subject.

12. A kit comprising:
(a) a lateral flow device comprising a first pad, a second pad, and a membrane, wherein the first pad receives a biological sample from a subject having a SARS-CoV2 infection, the second pad comprises an antibody that binds to an anti-ACE2 IgM antibody, and the membrane that reacts to the antibody bound to the anti-ACE2 IgM antibody; and
(b) instructions to use the kit to detect the anti-ACE2 IgM antibody in the biological sample.

13. The kit of claim 12, wherein the biological sample is a blood sample.

14. A lateral flow device adapted to perform the method of claim 3 for detecting an anti-ACE2-IgM antibody in a biological sample of a subject infected with a SARS-CoV2 virus.

15. The lateral flow device of claim 14, wherein the biological sample is a blood sample.

16. A dipstick assay to perform the method of claim 3 for detecting an anti-ACE2-IgM antibody in a biological sample of a subject infected with a SARS-CoV2 virus.

17. The dipstick assay of claim 16, wherein the biological sample is a blood sample.
